(19) United States
(12) Patent Application Publication
(10) Pub. No.: US 2015/0133495 A1
(43) Pub. Date: May 14, 2015

(54) Substituted Acyloxyamidines as HCV NS3/4A Inhibitors

(71) Applicant: ViroCura Therapeutics, Inc., Montreal (CA)

(72) Inventor: Daniel Lamarre, Montreal (CA)

(21) Appl. No.: 14/540,096

(22) Filed: Nov. 13, 2014

Related U.S. Application Data
(60) Provisional application No. 61/904,232, filed on Nov. 14, 2013.

(51) Int. Cl. C07D 417/12; C07C 251/68; C07D 285/06; C07D 261/18; C07D 413/14; C07D 307/68; C07D 413/12; C07D 317/68

(52) U.S. Cl. CPC C07D 417/12; C07D 413/12; C07C 251/68; C07D 317/68; C07D 261/18; C07D 413/14; C07D 307/68; C07D 285/06
USPC 514/314; 548/248; 514/378; 564/254; 514/640; 549/442; 514/466; 546/272.1; 514/340; 548/180; 514/367; 549/486; 514/471; 560/85; 514/533; 548/204; 514/365; 548/127; 514/361; 546/174; 562/440; 514/566; 435/375

(57) ABSTRACT

Disclosed herein is a compound of Formula I

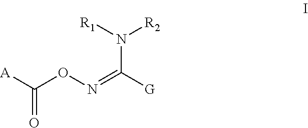

or a pharmaceutically acceptable salt thereof, in which A, G, R¹ and R² are as defined herein. The compounds and pharmaceutical compositions of the compounds are suitable for the treatment of HCV infection in mammals and are also useful to modulate or inhibit NS3/4 dimerization.

### CROSS REFERENCE TO RELATED APPLICATION(S)

Priority is hereby claimed to previously filed U.S. provisional patent application Ser. No. 61/904,232, filed on Nov. 14, 2013, the entire contents of which are hereby incorporated by reference.

### TECHNICAL FIELD

The present concerns substituted acyloxyamidine derivatives, their compositions and method of using same to inhibit the activity of HCV NS3/4A.

### BACKGROUND

Hepatitis C is a liver disease caused by the hepatitis C virus (HCV). About 200 million people are chronically infected with hepatitis C virus, and more than 350 000 people die from hepatitis C-related liver diseases each year. HCV infection is curable using increasingly effective antivirals and despite ongoing research, there is no vaccine on the horizon to prevent HCV infection. The therapeutic advance for chronic HCV infection will reside in combination treatment that includes different classes of HCV-specific inhibitors with synergistic antiviral potency, and more importantly with no overlapping resistance.

NS3 harbors three enzymatic activities: serine protease, NTPase and helicase. NS3 serine protease activity requires the co-factor NS4A (NS3/4A protease) and is responsible for the maturation of the NS proteins. The carboxy-terminal two thirds constitute the NS3 helicase belonging to the DExH family that is able to bind and to unwind in vitro RNA using the energy from ATP hydrolysis via its NTPase activity (1, 2). Although its exact biological function remains unclear, NS3 helicase has been shown to be absolutely necessary for HCV replication (3) and data from in vitro studies suggest that NS3 would track along double-stranded RNA and would unfold secondary structures in the vRNA and/or dsRNA intermediates (4,5). Importantly, it was proposed that NS3 oligomerization on RNA promotes the helicase processivity in vitro (6-8). In addition to its nucleic acid unwinding activity, it was recently proposed that NS3 via its helicase domain is involved in the regulation of HCV assembly process (as reported for other flaviviruses (9). Indeed, Ma et al. showed that Q221L mutation within HCV NS3 helicase domain was able to rescue the assembly and the infectivity of the chimeric clone HJ3 at a step following core/NS5A loading on lipid droplets (10). Interestingly, this function did not require the NS3 helicase activity per se. It was rather proposed that this function during assembly would be achieved through the interaction between NS3 helicase domain and host factors. Although both NS3 protease and helicase domains can function independently, it was demonstrated that these sub-domains can mutually regulate each other (11-13). Moreover, these activities can be modulated by other viral proteins such as NS5B (14, 15). Finally, NS3/4A protease has cellular substrates. Indeed through the cleavage of the adaptor proteins MAVS and TRIF, NS3/4A interferes with TLR3 and RIG-I signaling pathways, respectively, leading to the disruption of interferon-_ and interferon-stimulated genes transcription (16-20). This interference with host innate immunity confers an advantage to HCV infection. These studies highlight that NS3 is a multifunctional viral protein that harbors essential roles in several steps of HCV life cycle and whose regulation appears to be complex. With the discovery of N-terminus product inhibitors of NS3 protease (21, 22), rational drug design approaches were undertaken to develop selective HCV inhibitors with promise in blocking viral replication in infected patients. BILN 2061, discovered by Boehringer Ingelheim R&D Canada, was the first-in-class HCV inhibitor ever clinically successful in infected patients (23). First generation of direct-acting antivirals (DAA) INCIVEK®/Telaprevir (Vertex Pharmaceuticals) and Victrelis®/Boceprevir (Merck & Co) are NS3 protease inhibitors (PI) that were approved by the U.S. Food and Drug Administration (FDA) for use in the United States in mid-2011 (24). Despite efforts in the development of NS3 helicase inhibitors (25, 26) and progress made in the design of assays suitable for high throughput screening, no such inhibitors are currently evaluated in the clinic. Inhibition specificity and expected toxicity remain a challenge since the motor domain of NS3 helicase is similar to the one of many cellular proteins (27). Astex Pharmaceuticals has designed a novel class of NS3 PI that binds to an allosteric site at the NS3 protease/helicase interface, resulting in the stabilization of an inactive conformation and inhibition of the NS3 protease (28).

A successful therapy will most probably reside in the combination of multiple DAAs conferring a high barrier to resistance. Hence, disrupting interaction between viral proteins represent a very interesting avenue for the development of DAAs since viral escape would involve co-evolution of two HCV proteins, and significantly increasing the genetic barrier to resistance. The identification of small molecule inhibitors of HCV NS3/4A homotypic interactions that synergize with the first generation of NS3 protease inhibitors may provide significant advantages in a mechanism-based antiviral combination therapy for hepatitis C infection.

Thus, there is a need for improved pharmacological agents that are useful to treat HCV in humans.

### BRIEF SUMMARY

We have developed a drug discovery platform based on interaction of membrane proteins in live cell assays and applied the technology to the identification of small-molecule HCV inhibitors. As all HCV proteins encompass determinants responsible for their membrane anchoring, we completed a comprehensive HCV membrane protein-protein interaction (mPPI) analyses that are required in the virus life cycle. Many HCV proteins are anchored as monotopic membrane protein from one side and for which membrane-anchoring amphipathic_-helices (AH) are either involved in protein interaction and/or organelle localization. In a proof-of-principle drug discovery approach, we established the feasibility of High Throughput Screening (HTS) assays by implementing robust assays for selected HCV monotopic mPPIs. Prioritized assays were screened against a small-molecule sample collection (110,000 compounds and overall Z value >0.8) with self-association HCV proteins that targeted homodimeric interactions. By screening simultaneously many targets, hit compounds identified by their modulation of BRET signal in primary assay were prioritized based on selectivity as determined by the lack of activity obtained in the other cell-based BRET HTS assay. Hit compounds that were prioritized from the initial HTS screen demonstrated modulation of the BRET signal for NS3/4A dimer interactions and antiviral activity in a biologically relevant HCV replication model validated the mPPIs as valuable anti-HCV targets. The identified hit compounds for NS3/4A target had no effect on the serine protease activity of NS3/4A. Optimization of an antiviral compound series resulted in anti-HCV compounds with sub-micromolar potency and established a structure-activity relationship as shown by dose-response reduction of HCV RNA and disappearance of HCV proteins in a kinetic study. These data support HCV protein self-interaction as valuable anti-HCV targets.

Accordingly, there is provided a compound of Formula I

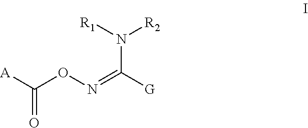

or a pharmaceutically acceptable salt thereof,
wherein
n is an integer of 1 or 2;

### A is

1) C₁-C₇ alkyl,
2) C₃-C₇ alkyl,
3) C₁-C₇ alkyl-(aryl)ₙ,

### 4) (R³)(R⁴)C,

### 5) (R⁵)(R⁶)(R⁷)C,

6) aryl,
7) heteroaryl,
8) heterocyclyl, or
9) biphenyl,
wherein the aryl is substituted with one or more R¹⁰ substituents and the biphenyl is optionally substituted with one or more R¹⁰ substituents;
wherein the heteroaryl is substituted with one or more R²⁰ substituents; and
wherein the heterocyclyl is optionally substituted with C₁-C₇ alkyl, aryl-C₁-C₇ alkyl, the aryl moiety being optionally substituted with one or more R¹⁰ substituents;

### G is

1) aryl,
2) C₁-C₇ alkyl-aryl,
3) heteroaryl, or
4) C₁-C₇ alkyl-heteroaryl,
wherein the aryl and the heteroaryl are optionally substituted with one or more R⁴⁰ substituents;
R¹ and R² are each independently

### 1) H,

2) C₁-C₇ alkyl,
3) C₃-C₇ cycloalkyl,
4) aryl,
5) heteroaryl, or
6) CH₂-heteroaryl,
wherein the aryl is optionally substituted with one or more R¹¹ substituents, and wherein the heteroaryl is optionally substituted with one or more R²⁰ substituents;
or
when R¹ is H, R² is covalently bonded to G to form a 3 to 7-membered heterocycle containing one or more heteroatoms selected from N, S or O;
R³, R⁴ when covalently bonded together form C₃-C₇ cycloalkyl or a heterocycle containing N or O heteroatoms optionally substituted with one or more R^{Y} substituents;
R⁵, R⁶ and R⁷ are each independently
1) C₁-C₇ alkyl,
2) C₃-C₇ cycloalkyl, or
3) aryl optionally substituted with one or more R^{X} substituents;
R⁸ and R⁹ are both independently

### 1) H,

2) C₁-C₇ alkyl,
3) C₃-C₇ cycloalkyl,
4) C₁-C₇ alkyl-C₃-C₇ cycloalkyl,
5) C(O)C₁-C₇alkyl,
6) C(O)C₃-C₇ cycloalkyl,

### 7) C(O)aryl,

### 8) C(O)heteroaryl

9) NHC(O)C₁-C₇ alkyl,
10) NHC(O)C₃-C₇ cycloalkyl,

### 11) NHC(O)aryl,

### 12) NHC(O)heteroaryl,

13) C₁-C₇ alkyl-aryl, or
14) C₁-C₇ alkyl-heteroaryl;

### R¹⁰ is

1) halo,

### 2) CN,

### 3) OH,

4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,

### 10) NR⁸R⁹,

### 11) NHC(O),

12) C(O)OC₁-C₇ alkyl,

### 13) C(O)OH, or

14) aryl optionally substituted with one or more with R¹¹ substituents;

### R¹¹ is

1) halo,

### 2) CN,

### 3) OH,

4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,

### 10) NR⁸R⁹;

### 11) NHC(O),

12) C(O)OC₁-C₇ alkyl, or

### 13) C(O)OH;

### R²⁰ is

1) C₁-C₇ alkyl,
2) C₃-C₇ cycloalkyl,
3) aryl optionally substituted with one or more R¹⁰ substituents,
4) C₁-C₇ alkyl-aryl,

### 5) CH₂OCH₃, or

6) heteroaryl optionally substituted with one or more R¹⁰ substituents,
7) aryl-C₁-C₇ alkyl substituted with one or more R¹⁰ substituents,
8) heteroaryl-C₁-C₇ alkyl substituted with one or more R¹⁰ substituents;
wherein the aryl is optionally substituted with a halo substituent;

### R⁴⁰ is

### 1) OR^{A},

2) halogen,
3) C₁-C₇ alkyl,
4) heteroaryl, or
5) haloalkane;

### R^{A} is

1) C₁-C₇ alkane,
2) C₁-C₆ alkyl-aryl,
3) aryl, or
4) C₁-C₇ alkyl-heteroaryl,
wherein the aryl and the heteroaryl are optionally substituted with a halo;

### R^{X} is

1) halo,
2) hydroxyl,
3) C₁-C₇ alkyl,
4) OC₁-C₇ alkyl; or
5) aryl optionally substituted with one or more Rz substituents;

### R^{Y} is

1) C₁-C₇ alkyl, or
2) aryl-C₁-C₇ alkyl optionally substituted with one or more R¹⁰ substituents; and

### R^{Z} is

1) C₁-C₇ alkyl,
2) halo,

### 3) OH,

4) OC₁-C₇ alkyl, or

### 5) NR⁸R⁹,

and with the proviso that the following compounds are excluded:

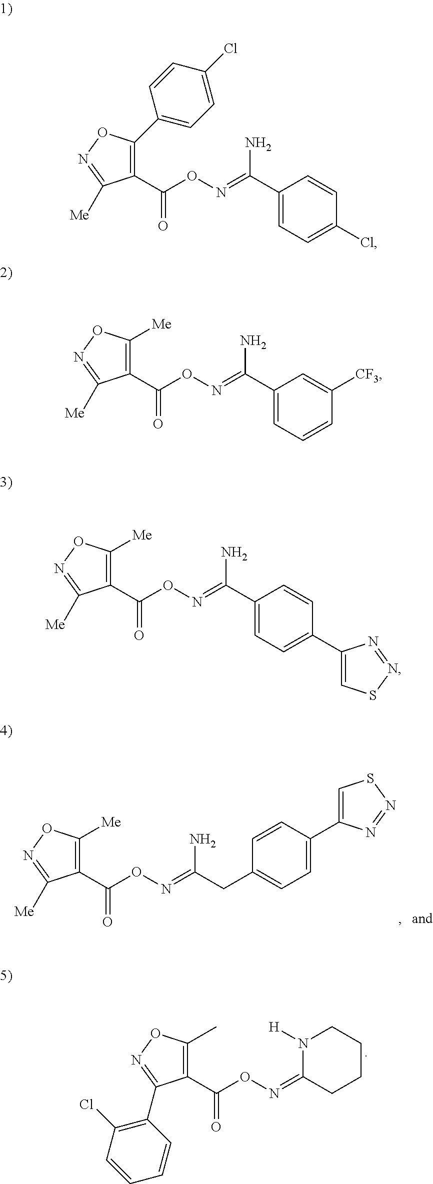 [SMILES: Cc1noc(-c2ccc(Cl)cc2)c1C(=O)O/N=C(\N)c1ccc(Cl)cc1 ; Cc1noc(C)c1C(=O)O/N=C(\N)c1cccc(C(F)(F)F)c1 ; Cc1noc(C)c1C(=O)O/N=C(\N)c1ccc(-c2csnn2)cc1 ; Cc1noc(C)c1C(=O)O/N=C(\N)Cc1ccc(-c2csnn2)cc1 ; Cc1onc(-c2ccccc2Cl)c1C(=O)O/N=C1/CCCCN1]

According to another aspect, there is provided a pharmaceutical composition comprising a compound of Formula 1 and a pharmaceutically acceptable carrier, without the provisos.

According to another aspect, there is provided a method of modulating or inhibiting dimerization of NS3/4A comprising: containing a cell infected by HCV with a compound, according to Formula I. so as to modulate or inhibit the dimerization, without the provisos.

According to another aspect, there is provided a method of treating HCV infection in a subject, the method comprising administering to a subject in need thereof a therapeutically effective amount of a compound of Formula I so as to treat the HCV infection, without the provisos.

### DETAILED DESCRIPTION

### 1) General

We have developed a drug discovery platform based on membrane protein-protein interactions (mPPIs) to identify novel class of HCV-specific mPPI inhibitors. We have demonstrated that NS3/4 dimerizes in live cells using bioluminescence resonance energy transfer (BRET) technology and co-immunoprecipitation method. In order to identify small molecule modulators of NS3/4A dimerization, we developed high throughput screening (HTS) cell-based BRET assays and screen a drug-like compound collection. Following the primary screen and a series of secondary assays, we identified hit structures and prioritized a lead inhibitor series having demonstrated dose-dependent modulation of NS3/4A dimerization and inhibition of vRNA replication in a sub-genomic HCV replicon assay. These data validate an allosteric mechanism for the requirement of NS3/4A protein dimerization or oligomerization as an essential step in the HCV life cycle and thus represent a valuable anti-HCV target. We have further shown that targeting NS3/4A dimer with lead compound does not affect NS3/4A protease activity.

Furthermore, the target site was validated in the HCV replicon by applying selective pressure with six analog compounds and allowing resistance mutations to emerge. HCV replicon resistant to potent inhibitors of lead series showed predominant variants encoding mutations in the NS3 protein of HCV genome, which have never been reported with existing class of NS3 protease active site inhibitors. The mutations are located at the subdomain of the helicase domain and accessible to intermolecular interactions. The low level of natural variants at the position of resistance mutations further supports a high genetic barrier of the target site. Thus compounds of the lead series represent novel first-in-class NS3 dimer interaction allosteric inhibitors.

### 2) Compounds

### Core:

In one subset, the Core is selected from one of the following Formulae 1A through 1H:

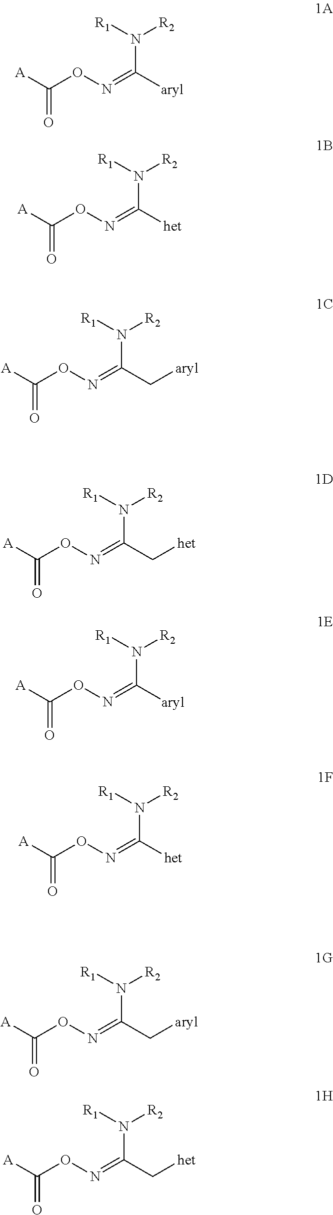

wherein the R¹, R², A, aryl and het are as defined herein

### A:

In one subset A is

1) C₁-C₇ alkyl,
2) C₃-C₇ alkyl,
3) C₁-C₇ alkyl-(aryl)ₙ,

### 4) (R³)(R⁴)C,

### 5) (R⁵)(R⁶)(R⁷)C,

6) aryl,
7) heteroaryl,
8) heterocyclyl, or
9) biphenyl,
wherein the aryl is substituted with one or more R¹⁰ substituents and the biphenyl is optionally substituted with one or more R¹⁰ substituents;
wherein the heteroaryl is substituted with one or more R²⁰ substituents; and
wherein the heterocyclyl is optionally substituted with C₁-C₇ alkyl, aryl-C₁-C₇ alkyl, the aryl moiety being optionally substituted with one or more R¹⁰ substituents.

In one example, A is phenyl optionally mono- or di- or tri-substituted with R¹⁰.

In another example, A is biphenyl or biphenyl mono- or di-substituted with R¹⁰.

In another example, A is naphtyl or naphtyl mono- or di-substituted with R¹⁰.

In one example, A is heteroaryl substituted with one or two R²⁰ substituents.

In one example, A is C₁-C₇ alkyl-(aryl)ₙ where n is 1 or 2.

### G:

In one subset, G is

1) aryl,
2) C₁-C₇ alkyl-aryl,
3) heteroaryl, or
4) C₁-C₇ alkyl-heteroaryl,

wherein the aryl and the heteroaryl are substituted with one or more R⁴⁰ substituents.

### R¹ and R²:

In one subset, R¹ and R² are each independently

### 1) H,

2) C₁-C₇ alkyl,
3) C₃-C₇ cycloalkyl,
4) aryl,
5) heteroaryl, or
6) CH₂-heteroaryl,
wherein the aryl is optionally substituted with one or more R¹¹ substituents, and wherein the heteroaryl is optionally substituted with one or more R²⁰ substituents; or

when R¹ is H, R² is covalently bonded to G to form a 3 to 7-membered heterocycle containing one or more heteroatoms selected from N, S or O.

In one example, R¹ and R² are both H.

In another example, R¹ is H and R² is C₁-C₇ alkyl.

In another example, R¹ and R² are both C₁-C₇ alkyl.

In another example, R¹ is H and R² is C₁-C₇ alkyl-heteroaryl.

### R³ and R⁴

In one subset, R³, R⁴ when covalently bonded together form C₃-C₇ cycloalkyl or a heterocycle containing N or O heteroatoms optionally substituted with one or more R^{Y} substituents.

### R⁵, R⁶ and R⁷:

In one subset, R⁵R⁶ and R⁷ are each independently

1) C₁-C₇ alkyl,
2) C₃-C₇ cycloalkyl, or
3) aryl optionally substituted with one or more R^{X} substituents;

### R⁸ and R⁹:

In one subset, R⁸ and R⁹ are both independently

### 1) H,

2) C₁-C₇ alkyl,
3) C₃-C₇ cycloalkyl,
4) C₁-C₇ alkyl-C₃-C₇ cycloalkyl,
5) C(O)C₁-C₇alkyl,
6) C(O)C₃-C₇ cycloalkyl,

### 7) C(O)aryl,

### 8) C(O)heteroaryl

9) NHC(O)C₁-C₇ alkyl,
10) NHC(O)C₃-C₇ cycloalkyl,

### 11) NHC(O)aryl,

### 12) NHC(O)heteroaryl,

13) C₁-C₇ alkyl-aryl, or
14) C₁-C₇ alkyl-heteroaryl.

### R¹⁰:

In one subset, R¹⁰ is

1) halo,

### 2) CN,

### 3) OH,

4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹ mono or bis lower alkyl amino;

### 11) NHC(O),

12) C(O)OC₁-C₇ alkyl, or

### 13) C(O)OH, or

13) aryl optionally substituted with one or more with R¹¹ substituents.

### R¹¹:

In one subset, R¹¹ is

1) halo,

### 2) CN,

### 3) OH,

4) C₁-C₇ alkyl
5) C₃-C₇ cycloalkyl,
6) OC₁-C₇ alkyl,
7) SC₁-C₇ alkyl,
8) haloalkyl,
9) C₁-C₇ alkyl-aryl,

### 10) NR⁸R⁹;

### 11) NHC(O),

12) C(O)OC₁-C₇ alkyl, or

### 13) C(O)OH.

### R²⁰:

In one subset, R²⁰ is

1) C₁-C₇ alkyl,
2) C₃-C₇ cycloalkyl,
3) aryl optionally substituted with one or more R¹⁰ substituents,
4) C₁-C₇ alkyl-aryl,

### 5) CH₂OCH₃, or

6) heteroaryl optionally substituted with one or more R¹⁰ substituents,
7) aryl-C₁-C₇ alkyl substituted with one or more R¹⁰ substituents,
8) heteroaryl-C₁-C₇ alkyl substituted with one or more R¹⁰ substituents;
wherein the aryl is optionally substituted with a halo substituent;

### R⁴⁰:

In one subset, R⁴⁰ is

### 1) OR^{A},

2) halogen,
3) C₁-C₇ alkyl,
4) heteroaryl, or
5) haloalkane.

### R^{A}:

In one subset, R^{A} is

1) C₁-C₇ alkyl,
2) C₁-C₆ alkyl-aryl,
3) aryl, or
4) C₁-C₇ alkyl-heteroaryl,
wherein the aryl and the heteroaryl are optionally substituted with a halo.

### R^{X}:

In one subset, R^{X} is

1) halo,
2) hydroxyl,
3) C₁-C₇ alkyl,
4) OC₁-C₇ alkyl; or
5) aryl optionally substituted with one or more R^{Z} substituents.

### R^{Y}:

In one subset, R^{Y} is

1) C₁-C₇ alkyl, or
2) aryl-C₁-C₇ alkyl optionally substituted with one or more R¹⁰ substituents.

### R^{Z}:

In one subset, R^{Z} is

1) C₁-C₇ alkyl,
2) halo,

### 3) OH,

4) OC₁-C₇ alkyl, or

### 5) NR⁸R⁹.

### DEFINITIONS

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “alkyl” is intended to include both branched and straight chain saturated aliphatic hydrocarbon groups having the specified number of carbon atoms, for example, for example, C₁-C₇ as in C₁-C₇ alkyl is defined as including groups having 1, 2, 3, 4, 5, 6 or 7 carbons in a linear or branched arrangement. Examples of C₁-C₇ alkyl as defined above include, but are not limited to, methyl, ethyl, n-propyl, i-propyl, n-butyl, t-butyl, i-butyl, pentyl, hexyl, heptyl, 1-methylethyl, 1-methylpropyl, 2-methylpropyl, 1,1-dimethylethyl, 1,1,2,2-tetramethylpropyl.

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3, 4, 5, 6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl.

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “lower alkoxy” is intended to mean OC₁-C₇ alkyl and includes methoxy, ethoxy, propoxy, 1-methylethoxy, n-butoxy, 1-methylpropoxy, and 1,1-dimethylethoxy (commonly referred as tert-butoxy).

As used herein, the term “lower thioalkyl” is intended to mean SC₁-C₇ alkyl and includes methylthio, ethylthio, propylthio, 1-methylethethio, n-butylthio, 1-methylpropylthio, and 1,1-dimethylethylthio.

As used herein, the term “amino” as used herein means an amino radical of formula —NH₂. The term “lower alkylamino” as used herein means alkylamino radicals containing one to seven carbon atoms and includes methylamino, ethylamino, propylamino, (1-methylethyl)amino and 2-methylbutyl)amino. The term “di(lower alkyl)amino” means an amino radical having two lower alkyl substituents each of which contains one to seven carbon atoms and includes dimethylamino, diethylamino, ethylmethylamino and the like.

As used herein, the term “aryl”, either alone or in combination with another radical, means a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, 1-naphthyl, 2-naphthyl and tetrahydronaphthyl. The aryls may be connected to another group either at a suitable position on the cycloalkyl ring or the aromatic ring

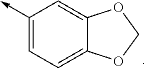

As used herein, the term “biphenyl” is intended to mean two phenyl groups covalently bonded together. One example of such a biphenyl is

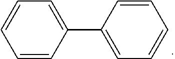 [SMILES: c1ccc(-c2ccccc2)cc1]

As used herein, the term “heteroaryl”, “Het” or “het” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Optionally, the heteroaryl may bear one or two substituents; for example, N-oxydo, lower alkyl (C₁-C₆ alkyl), cycloalkyl (C₃-C₇ cycloalkyl), lower alkoxy (OC₁-C₆ alkyl), lower thioalkyl (SC₁-C₆ alkyl), phenyl lower alkyl, halo, cyano (CN), amino or mono- or di-lower alkylamino. Again optionally, the five- or six-membered heterocycle can be fused to a second cycloalkyl, an aryl (eg: phenyl) or another heterocycle. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, and fluoroscein derivatives.

As used herein, the term “heterocycle”, “heterocyclic” or “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl, imidazolidinyl, morpholinyl, imidazolinyl, pyrazolidinyl, pyrazolinyl.

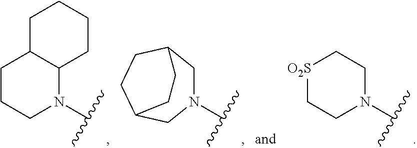 [SMILES: *N1CCCC2CCCCC21 ; *N1CC2CCC(CC2)C1 ; *N1CCS(=O)(=O)CC1]

Examples of suitable heterocycles and optionally substituted heterocycles include morpholine, pyrrolidine, piperidine, 1-methylpiperidine, piperazine, 1-methylpiperazine, 1,4-dioxane, tetrahydrofuran, furan, thiophene, pyrazole, pyrrole, 1H-imidazole, thiazolidine, 1-methylimidazole, oxazole, isoxazole, thiazole, 2-methylthiazole, 2-aminothiazole, 2-(acetylamino)thiazole, 2-(methylamino)thiazole, thiadiazole, 1H-tetrazole, 1-methyl-1H-tetrazole, 2-methyl-2H-tetrazole, pyridine, pyridine N-oxide, pyrimidine, 2,4-dimethylpyrimidine, 2,6-dimethylpyridine, quinoline, isoquinoline, 3,4-methylene-dioxyphenyl, 4,5-methylene-dioxyphenyl, benzofuran, indole, indazole, benzimidazole.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

If the substituents themselves are incompatible with the synthetic methods described herein, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (4th ed.), John Wiley & Sons, NY (2007), which is incorporated herein by reference in its entirety. Examples of protecting groups used throughout include, but are not limited to Fmoc, Bn, Boc, CBz and COCF₃. In some instances, a substituent may be specifically selected to be reactive under the reaction conditions used in the methods described herein. Under these circumstances, the reaction conditions convert the selected substituent into another substituent that is either useful in an intermediate compound in the methods described herein or is a desired substituent in a target compound.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

The compounds of Formula I, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)- or (S)- or, as (D)- or (L)- for amino acids. The present is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of Formula I may exist as a mix of epimers. Epimers means diastereoisomers that have the opposite configuration at only one of two or more stereogenic centres present in the respective compound.

Certain compounds of Formula I may exist in Zwitterionic form and the present includes Zwitterionic forms of these compounds and mixtures thereof.

In addition, the compounds of Formula I also may exist in hydrated and anhydrous forms. Hydrates of the compound of any of the formulas described herein are included. In a further embodiment, the compound according to any of the formulas described herein is a monohydrate. In one embodiment, the compounds described herein comprise about 10% or less, about 9% or less, about 8% or less, about 7% or less, about 6% or less, about 5% or less, about 4% or less, about 3% or less, about 2% or less, about 1% or less, about 0.5% or less, about 0.1% or less by weight of water. In another embodiment, the compounds described herein comprise, about 0.1% or more, about 0.5% or more, about 1% or more, about 2% or more, about 3% or more, about 4% or more, about 5% or more, or about 6% or more by weight of water.

The term “therapeutically effective amount” means an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

As used herein the term “treatment” as used herein is intended to mean the administration of a compound or composition according to the present invention to alleviate or eliminate symptoms of the hepatitis C disease and/or to reduce viral load in a patient. The term “treatment” also encompasses the administration of a compound or composition according to the present invention post-exposure of the individual to the virus but before the appearance of symptoms of the disease, and/or prior to the detection of the virus in the blood, to prevent the appearance of symptoms of the disease and/or to prevent the virus from reaching detectable levels in the blood.

As used herein, the term “treating HCV” is intended to mean the administration of a pharmaceutical composition described herein to a subject, preferably a human, which is afflicted with HCV to cause an alleviation of the HCV symptoms.

As used herein, the term “subject” is intended to mean mammal and includes humans, as well as non-human mammals which are susceptible to infection by hepatitis C virus. Non-human mammals include but are not limited to domestic animals, such as cows, pigs, horses, dogs, cats, rabbits, rats and mice, and non-domestic animals.

As used herein, the term “IC₅₀” is intended to mean an amount, concentration or dosage of a particular compound of Formula I that achieves a 50% inhibition of a maximal agonist response.

As used herein, the term “EC₅₀” is intended to mean an amount, concentration or dosage of a particular compound of Formula I that achieves a 50% of its maximal effect.

### 3. Utilities

The compounds as described herein are useful as inhibitors or modulators of HCV NS3/4A dimerization and as such the compounds, compositions and methods described herein include application to the cells or subjects afflicted with a particular disease state, which is mediated by a HCV. Thus, the compounds, compositions and methods are used to treat HCV infection in mammals, particularly humans.

The treatment involves administration to a subject in need thereof a compound of Formula I or a pharmaceutically acceptable salt thereof, or a pharmaceutical composition comprising a pharmaceutical carrier and a therapeutically effective amount of a compound of Formula I, or a pharmaceutically acceptable salt thereof. In particular, the compounds, compositions and methods described herein are useful in the treatment of HCV infection.

The compounds described herein, or their pharmaceutically acceptable salts or their prodrugs, may be administered in pure form or in an appropriate pharmaceutical composition, and can be carried out via any of the accepted modes of Galenic pharmaceutical practice.

The pharmaceutical compositions described herein can be prepared by mixing a compound of described herein with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral (subcutaneous injections, intravenous, intramuscular, intrasternal injection or infusion techniques), sublingual, ocular, rectal, vaginal, and intranasal. Pharmaceutical compositions described herein are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound described herein in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound described herein, or a pharmaceutically acceptable salt thereof, for treatment of a disease-state as described above.

A pharmaceutical composition described herein may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example inhalatory administration.

For oral administration, the pharmaceutical composition is typically in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When used for oral administration, a typical composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions described herein, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; encapsulating agents such as cyclodextrins or functionalized cyclodextrins; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is typically sterile.

A liquid pharmaceutical composition may be used for either parenteral or oral administration should contain an amount of a compound described herein such that a suitable dosage will be obtained. Typically, this amount is at least 0.01% of a compound described herein in the composition. When intended for oral administration, this amount may be varied to be between 0.1 and about 70% of the weight of the composition. For parenteral usage, compositions and preparations described herein are prepared so that a parenteral dosage unit contains between 0.01 to 10% by weight of the compound described herein. Pharmaceutical compositions may be further diluted at the time of administration; for example a parenteral formulation may be further diluted with a sterile, isotonic solution for injection such as 0.9% saline, 5 wt % dextrose (D5W), Ringer's solution, or others. The pharmaceutical composition described herein may be used for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound described herein from about 0.1 to about 10% w/v (weight per unit volume). The pharmaceutical composition described herein may be used for rectal administration of a suppository, which will melt in the rectum and release the drug. The composition for rectal administration may contain an oleaginous base as a suitable nonirritating excipient. Such bases include, without limitation, lanolin, cocoa butter and polyethylene glycol. The pharmaceutical composition described herein may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition described herein in solid or liquid form may include an agent that binds to the compound described herein and thereby assists in the delivery of the compound. Suitable agents that may act in this capacity include, but are not limited to, a monoclonal or polyclonal antibody, a protein or a liposome.

The pharmaceutical composition described herein may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds described herein may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions described herein may be prepared by methodology well known in the pharmaceutical art. For example, a pharmaceutical composition intended to be administered by injection can be prepared by mixing a compound described herein with sterile, distilled water so as to form a solution. A surfactant may be added to facilitate the formation of a homogeneous solution or suspension. Surfactants are compounds that non-covalently interact with the compound described herein so as to facilitate dissolution or homogeneous suspension of the compound in the aqueous delivery system.

The compounds described herein, or their pharmaceutically acceptable salts, may be administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of a compound described herein, or a pharmaceutically acceptable salt thereof.

### 4. Screening Assays

The compounds described herein may also be used in a method to screen for other compounds that modulate or prevent dimerization of HCV NS3/4A proteins. Generally speaking, to use the compounds described herein in a method of identifying compounds that modulate or prevent HCV NS3/4A dimerization, a monomer of NS3/4A is bound to a support, and a compound described herein is added to the assay. Alternatively, the compound may be bound to the support and the monomer is added.

There are a number of ways in which to determine the binding of a compound described herein to the NS3/4A monomer. In one way, the compound, for example, may be fluorescently or radioactively labeled and binding determined directly. For example, this may be done by attaching the monomer to a solid support, adding a detectably labeled compound, washing off excess reagent, and determining whether the amount of the detectable label is that present on the solid support. Numerous blocking and washing steps may be used, which are known to those skilled in the art.

In some cases, only one of the components is labeled. For example, specific residues in the monomer may be labeled. Alternatively, more than one component may be labeled with different labels; for example, using ¹²⁵I for the monomer, and a fluorescent label for the probe.

The compounds described herein may also be used as competitors to screen for additional drug candidates or test compounds. As used herein, the terms “drug candidate” or “test compounds” are used interchangeably and describe any molecule, for example, protein, oligopeptide, small organic molecule, polysaccharide, polynucleotide, and the like, to be tested for bioactivity. The compounds may be capable of directly or indirectly altering the HCV NS3/4 biological activity.

Drug candidates can include various chemical classes, although typically they are small organic molecules having a molecular weight of more than 100 and less than about 2,500 Daltons. Candidate agents typically include functional groups necessary for structural interaction with proteins, for example, hydrogen bonding and lipophilic binding, and typically include at least an amine, carbonyl, hydroxyl, ether, or carboxyl group. The drug candidates often include cyclical carbon or heterocyclic structures and/or aromatic or polyaromatic structures substituted with one or more functional groups.

Drug candidates can be obtained from any number of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Competitive screening assays may be done by combining an NS3/4 monomer and a probe to form a probe:monomer complex in a first sample followed by adding a test compound from a second sample. The binding of the test is determined, and a change or difference in binding between the two samples indicates the presence of a test compound capable of preventing or modulating dimerization.

In one case, the binding of the test compound can be determined through the use of competitive binding assays. In this example, the probe is labeled with a fluorescent label. Under certain circumstances, there may be competitive binding between the test compound and the probe. Test compounds which display the probe, resulting in a change in fluorescence as compared to control, are considered to bind to the monomer.

In one case, the test compound may be labeled. Either the test compound, or a compound described herein, or both, is added first to the NS3/4 monomer for a time sufficient to allow binding to form a complex.

Formation of the probe:monomer complex typically require Incubations of between 4° C. and 40° C. for between 10 minutes to about 1 hour to allow for high-throughput screening. Any excess of reagents are generally removed or washed away. The test compound is then added, and the presence or absence of the labeled component is followed, to indicate binding to the monomer.

In one case, the probe is added first, followed by the test compound. Displacement of the probe is an indication the test compound is binding to the NS3/4 monomer and thus is capable of binding to, and potentially modulating, the formation of a dimer. Either component can be labeled. For example, the presence of probe in the wash solution indicates displacement by the test compound. Alternatively, if the test compound is labeled, the presence of the probe on the support indicates displacement.

In one case, the test compound may be added first, with incubation and washing, followed by the probe. The absence of binding by the probe may indicate the test compound is bound to the NS3/4 monomer with a higher affinity. Thus, if the probe is detected on the support, coupled with a lack of test compound binding, may indicate the test compound is capable of binding to the monomer.

Modulation is tested by screening for a test compound's ability to modulate the dimerization of NS3/4 and includes combining a test compound with the monomer, as described above, and determining an alteration in the biological activity of the monomer. Therefore in this case, the test compound should bind to the monomer or dimer, and prevent or modulate dimerization and alter the biological activity of NS 3/4A.

Positive controls and negative controls may be used in the assays. All control and test samples are performed multiple times to obtain statistically significant results. Following incubation, all samples are washed free of non-specifically bound material and the amount of bound probe determined. For example, where a radiolabel is employed, the samples may be counted in a scintillation counter to determine the amount of bound compound.

Typically, the signals that are detected in the assay may include fluorescence, resonance energy transfer, time resolved fluorescence, radioactivity, fluorescence polarization, plasma resonance, or chemiluminescence and the like, depending on the nature of the label. Detectable labels useful in performing screening assays described herein include a fluorescent label such as Fluorescein, Oregon green, dansyl, rhodamine, tetramethyl rhodamine, Texas Red, Eu³⁺; a chemiluminescent label such as luciferase; colorimetric labels; enzymatic markers; or radioisotopes such as tritium, I¹²⁵ and the like

Affinity tags, which may be useful in performing the screening assay described herein include be biotin, polyhistidine and the like.

### 5. Synthesis and Methodology

General methods for the synthesis of the compounds are shown below and are disclosed merely for the purpose of illustration and are not meant to be interpreted as limiting the processes to make the compounds by any other methods. Those skilled in the art will appreciate that a number of methods are available for the preparation of compounds described herein.

Schemes 1 to 11 illustrate general synthetic procedures for the preparation of compounds described herein. Scheme 1 describes a general synthetic approach to the compounds described herein. Compounds 1-ix are prepared by the following sequence. Dihalogenated compounds such as 1-i are treated with an amine 1-ii at room temperature to provide the monohalogenated compounds 1-iii. Reduction to the intermediates 1-iv is carried out using a number of methods known by those skilled in the art. Intermediates 1-iv are treated with electrophilic compounds 1-v in the presence of a coupling, dehydrating or oxidizing agent to yield the imidazopyridine intermediates 1-vi. The compounds described herein e.g. 1-ix are finally obtained by treatment with nucleophiles 1-vii under various carbonylation methods known by those skilled in the art.

Table I

IC₅₀ value is the half maximal concentration of the tested compound to inhibit half of the maximal response of the HCV RNA replication. It is a measurement of the antiviral compound potency. At day 1, 10,000 human hepatoma Huh7 cells containing the HCV replicon are added to 96-well plates. At day 3, compounds are added to the cells with various concentrations of compounds [0.01 uM to 10 μM]. At day 5, the luciferase reporter activities is determined as a surrogate of HCV RNA replication

Curve Fitting

All curve fitting was conducted using non-linear regression analyses from PRISM (version 4.0c, GraphPad Inc.) and the determination of Rmax and EC₅₀ or IC₅₀ parameters were obtained from sigmoidal dose-response phase (variable slope) equation.

### Bioloqy

### Cell-Based BRET HTS Assays

Cell-based High Throughput Screening (HTS)-compatible Bioluminescence Resonance Energy Transfert (BRET) assays were used to identify modulators of HCV membrane protein-protein interactions. HTS BRET assays performed in 384-well plates were screened against a small-molecule sample collection with self-association HCV proteins that targeted the homodimeric NS3/4A interaction. The overall Z value indicative of the reproducibility of the cell-based BRET assay was excellent with value >0.8. From the screening data, the average of all compounds tested for NS3/4A dimer in BRET signal percentage of inhibition was 4%±18.6%. Based on the average activity values, the cut-off for the identification of hit compounds was established at 50% of the control (˜2.5 times the standard deviation). By screening simultaneously many targets, hit compounds identified by their modulation of BRET signal in primary assay were prioritized based on selectivity as determined by the lack of activity obtained in other cell-based BRET HTS assay. Hit compounds were prioritized from the initial HTS screen with significant modulation of BRET signal of NS3/4A dimer interaction and hit compounds were then evaluated in the biologically relevant model of HCV replicon for identification of antiviral compounds. A selective lead series of compounds for NS3/4A dimer interaction having demonstrated inhibition of HCV replication in the sub-genomic HCV replicon assay with EC₅₀ in the micromolar range were selected for optimization. The identified antiviral compounds targeting the NS3/4A dimer interaction had no effect on the protease activity of NS3/4A.

### HCV Replicon

The replicon HCV1b is generated based on the wild-type sequence CON-1 genotype b (GenBank accession number AJ238799; Lohmann et al., Science 1999, 285: 110-113) and expressed the sub-genomic fragment HCV NS3-NS4A-NS4B-NS5A-NS5B. The replicon contain a hybrid HCV-IRES 5′UTR, a modified luciferase reporter gene expressed as a luciferase-Ubi-neomycin phosphotransferase gene fusion and an EMCV IRES NS3-NS5B subgenomic fragment with its 3′UTR. To generate cell lines harboring the replicon, Huh-7 cells are electroporated with purified in vitro HCV1b RNA transcripts and stable cell lines are selected in the presence of G418. Stable replicon cell lines are established as described in Science 1999, 285: 110-113. The amount of luciferase expressed by selected cells directly correlates with the level of HCV RNA replication, as measured by real-time PCR.

### HCV Replicon RNA Replication Assay

Stable HCV replicon cells are maintained in Dulbecco's Modified Eagle Medium (DMEM) supplemented with 10% FBS and 0.5 mg/ml G418. During the assay, DMEM supplemented with 10% FBS, containing 0.5% DMSO and lacking G418 are used as assay medium. For the assay, cell stocks are trypsinized and diluted in assay medium to distribute 7,500 cells in 96-well plates. The plates are then incubated at 37° until compound addition. Serial dilutions of the test compound are prepared in 100% DMSO, before dilution in assay medium to a final DMSO concentration of 0.5% to generate a concentration dose response curves. A fixed volume from each well of the compound dilution plate is transferred to a corresponding well of the cell culture plate. The cell culture plate is incubated at 37° C. with 5% CO₂ for 72 hours. Following the 72 h incubation period, the medium is aspirated from the 96-well assay plate and a volume of 50_l of lysis buffer 100 mM Tris acetate, 20 mM Mg acetate, 2 mM EGTA, 3.6 mM ATP, 1% Brij 58, 0.7%_-mercaptoethanol containing 45_g/ml luciferine (pH 7.9) is added to each well. The luciferase activity is determined using the luciferase substrate and the luminescence is detected on a MicroBeta JET 1450 LSC & Luminescence Counter (Perkin Elmer) instrument. The luminescence in each well of the culture plate is a measure of the amount of HCV RNA replication in the presence of various concentrations of inhibitor. The % inhibition is calculated for each inhibitor concentration and used to determine the concentration that results in 50% inhibition of HCV replication (EC₅₀).

### Chemistry

The following examples illustrate further this invention. As general guidelines, solution percentages or ratios express a volume to volume relationship, unless stated otherwise. Temperatures are given in degrees Celsius. Proton (₁H) nuclear magnetic resonance (NMR) spectra were recorded on either a Varian 400-MR (400 MHz) or a Varian INOVA-600 (600 MHz) spectrometer and the chemical shifts (∂) are reported in parts per million (ppm). HPLC (High Performance Liquid Chromatography) instrument used was an Agilent model 1200 series with either a G1365 VWD UV or 1260 ELSD G4218A detector. Compound homogeneity is expressed in % by reference to the total UV (ultraviolet) absorbance of the various components of the sample. It is measured by UV detection based on HPLC analysis. HPLC conditions:

Column 1: Sunfire C18 3.5 microns 4.6×30 mm; Column 2: Zorbax XDB-C18, 4.6×30 mm: using Solv. A Water:MeOH:TFA (95:5:0.05) and solvent B Water:MeOH:TFA (5:95:0.05) with a gradiant 0% A to 100% B over 6 min then 100% B.

HPLC-MS (ESI or APCI) was conducted on an Agilent 6120 Quadrupole equipped with a 1260 Infinity LC component and a 1260 DAD (UV detector). HRMS was obtained with an Agilent G1969A ToF instrument using an Agilent 1100 series LC component and the following conditions: Zorbax XBD-C18 2.1×30 mm, 3.5 um using Solvent A Water:ACN:HCO₂H (95:5:0.05) and Solvent B Water:ACN:HCO₂H (5:95:0.05) 0% A to 100% B over 3.5 min then 1 min 100% B.

Flash chromatography was generally conducted using an ISCO system with RediSep Gold silica columns and the product eluted with an appropriate gradient of solvents to produce different fractions which were pooled according to the desired homogeneity and the solvent evaporated to afford the purified product. The abbreviations used in the examples include Me: methyl; Et: ethyl; Bzl: benzyl; t-Bu: tert-butyl; ACN: acetonitrile; DCM: dichloromethane; DMF: N,N-dimethylformamide; DIPEA: diisopropylethylamine; Et₂O: diethylether; EtOAc: ethyl acetate; EtOH: ethanol; MeOH; methanol; Ph: phenyl; THF: tetrahydrofuran; NCS: N-chlorosuccinimide; DMAP: 4-dimethylaminopyridine; etc

### Example 1

### (Z)—N′-((3-(2-chlorophenyl)isoxazole-4-carbonyl)oxy)benzo[d][1,3]dioxole-5-carboximidamide obtained by the following synthetic sequence from Intermediates 1 to 5

### Intermediates 1:

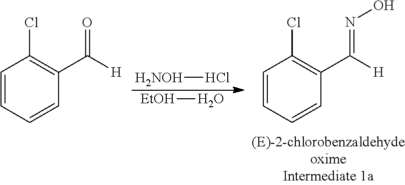

A mixture of 2-chlorobenzaldehyde (1.6 mL, 14.23 mmol) and hydroxylamine hydrochloride (1.0 g, 14.51 mmol) in ethanol (1.5 mL), water (4.50 mL) and ice (8.00 mL) to give a white suspension was cooled to 0° C. and aq. sodium hydroxide 50% wt. (1.61 mL, 31.3 mmol) was added dropwise and then allowed to warm to rt. After 0.5 hour, the mixture was cooled to 0° C., acidified by dropwise addition of conc. HCl (1.484 ml, 18.07 mmol) and the resulting off-white slurry was stirred at 0° C. for 30 minutes. The resulting off-white solid was filtered, washed with water (2×5 mL) and at dried at 20° C. under high vacuum until constant weight to give (E)-2-chlorobenzaldehyde oxime 1a (1.98 g, 89% yield) as a yellow solid MS, m/z 156.0 (MH+); HPLC 93.8%, RT=1.87 min.

### Intermediates 2:

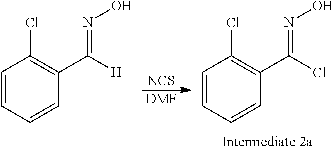

NCS (1.954 g, 14.64 mmol) was added portionwise over 60 minutes to a solution of (E)-2-chlorobenzaldehyde oxime (1.98 g, 12.73 mmol) in DMF (10.59 ml) to give a colorless solution (Exotherm noted after 10 min and 20% NCS added). After 1 h 45, the reaction was deemed completed by TLC and then poured into 40 mL of water. The mixture was then extracted with Et₂O (2×40 mL) and the combined organic layers were washed with water (3×30 mL) and brine (30 mL). The organic layer was dried over anh. MgSO₄, filtered and concentrated to dryness to afford (Z)-2-chloro-N-hydroxybenzimidoyl chloride 2a (2.31 g, 96% yield) as a yellow oil: MS, m/z 186.1 (M-Cl+MeO); HPLC 95.8%, RT=1.86 min.

### Intermediates 3:

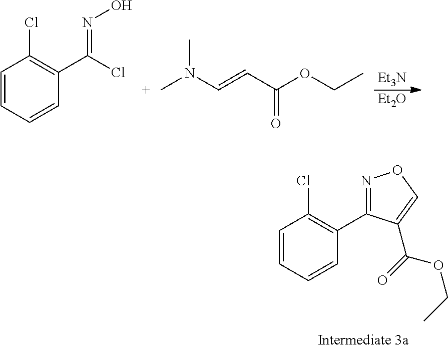

A solution of ethyl 3-(dimethylamino)acrylate (4.33 mL, 30.3 mmol) and triethylamine (2.446 mL, 17.55 mmol) in Et₂O (46 mL) was added dropwise to a solution of (Z)-2-chloro-N-hydroxybenzimidoyl chloride (2.3 g, 12.10 mmol) in Et₂O (46.0 mL) to give a white suspension which was stirred overnight at 20° C. After 19 h, the salts were filtered and then rinsed with Et₂O (30 mL). The filtrate was concentrated to dryness to give 5.86 g of an orange liquid which was purified by flash chromatography using a mixture of hexane/Et₂O to afford ethyl 3-(2-chlorophenyl)isoxazole-4-carboxylate 3a (2.36 g, 77% yield) as a colorless oil; 1H NMR (DMSO-d₆) was consistent with the desired product; MS m/z 252.1 (MH⁺); HPLC 100%, Rt=1.96 min, column 2.

Alternatively, the desired 5-substituted isoxazole-4-carboxylate derivative can be obtained using a 3-substituted-3-oxoproprionate reagent using the following procedure:

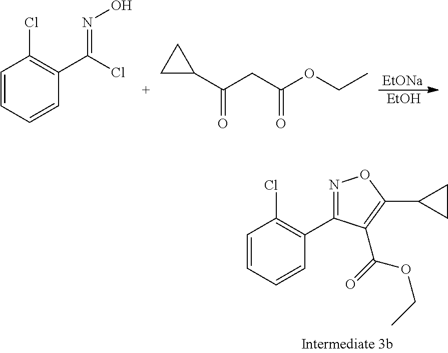

A solution of sodium ethoxide (21 wt. % in EtOH (3.71 mL, 9.95 mmol)) was added to ethyl 3-cyclopropyl-3-oxopropanoate (1.553 g, 9.95 mmol) in EtOH (7.57 mL) and the resulting mixture was cooled to 0° C. and stirred for 30 minutes. This solution was then added to a cold (0° C.) solution of (Z)-2-chloro-N-hydroxybenzimidoyl chloride (1.8 g, 9.47 mmol) in EtOH (10.00 ml) over 45 minutes the allowed to warm to 20° C. and stirred for 17 h. The mixture was the concentrated and partitioned between Et2O (50 mL) and water (50 mL). The layers were separated and the aqueous layer was back-extracted with Et₂O (25 mL). The combined organic layers were washed with brine (25 mL), dried over anh. MgSO₄, filtered and concentrated to give 2.50 g as a yellow oil which was purified by flash chromatography to give ethyl 3-(2-chlorophenyl)-5-cyclopropylisoxazole-4-carboxylate as intermediate 3b (1.91 g, 69.1% yield) as a colorless oil: ¹H NMR (400 MHz, DMSO-d₆)_ppm 0.98 (t, J=7.04 Hz, 3H) 1.22-1.28 (m, 2H) 1.28-1.34 (m, 2H) 2.78-2.88 (m, 1H) 4.07 (q, J=7.04 Hz, 2H) 7.43-7.50 (m, 2H) 7.52-7.58 (m, 1H) 7.58-7.62 (m, 1H); MS m/z 292.1 (MH+); HPLC >98%, column 2, Rt=2.16 min.

### Intermediates 4:

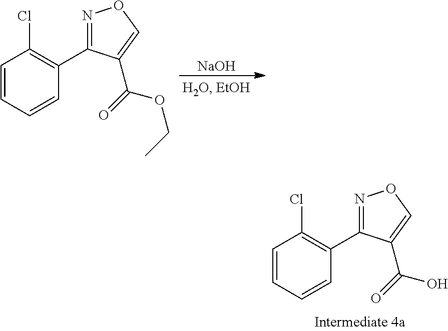

To a solution of ethyl 3-(2-chlorophenyl)isoxazole-4-carboxylate (2.35 g, 9.34 mmol) in EtOH (12.92 mL) was added 4M aq. NaOH (2.6 mL, 10.27 mmol) to give a yellow solution which was heated to reflux (80° C.) for 1 h. The mixture was then cooled to 0° C., acidified with dropwise addition of HCl 2N (5.14 mL, 10.27 mmol) and water (15 mL) but the desired compound did not crystallize. The solution was then basified by addition of NaOH 4M (2.57 ml, 10.27 mmol) and washed with Et₂O (2×15 mL) and the combined organic layers were discarded. The aqueous layer was acidified with 2N HCl (5.14 mL) and then extracted with Et₂O (2×20 mL). The combined organic layers were washed with brine (15 mL), then dried over anh.MgSO₄, filtered and concentrated to give 850 mg as an orange oil which was purified by Flash chromate (Hex/EtOAc) to afford 3-(2-chlorophenyl)isoxazole-4-carboxylic acid 4a (429 mg, 20.55% yield) as a yellow solid. ¹H NMR (DMSO-d₆) was consistent with the desired product: MS m/z 224.0107 (MH⁺); HPLC 100%, Rt=1.61 min.

### Intermediates 5:

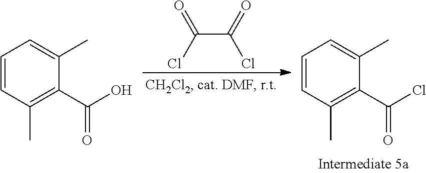

Oxalyl chloride (0.105 mL, 1.199 mmol) was added dropwise to a solution of 2,6-dimethylbenzoic acid (150 mg, 0.999 mmol) in DCM (6 mL) and the reaction was stirred at room temperature overnight. Solvents were evaporated giving 140 mg (83%) of crude acid chloride as a white solid (Intermediate 5a) and was used directly in the next step in the preparation of Example 2.

Intermediate 5b: A solution of dibenzo[c,e]oxepine-5,7-dione (1 g, 4.46 mmol) in iso-propyl alcohol (8.00 mL) was heated to reflux (82° C.) for 43 h then cooled to 20° C. and the solvent was evaporated to dryness under reduced pressure to give a colorless oil. The residue was purified by flash chromatography to give 2′-(isopropoxycarbonyl)-[1,1′-biphenyl]-2-carboxylic acid (970 mg, 3.41 mmol, 76% yield) as a white solid (¹H NMR in DMSO-d₆ was consistent with the expected product): MS m/z 283.1 (M-H—); HPLC 100%, Rt 2.05 min, column 2.

Oxalyl chloride (0.269 mL, 3.08 mmol) was added followed by DMF (9.53 μL, 0.123 mmol) to a solution of 2′-(isopropoxycarbonyl)-[1,1′-biphenyl]-2-carboxylic acid (0.35 g, 1.231 mmol) in DCM (5 mL) to give a colorless solution. Following gas evolution and stirring at room temperature for 1.5 h, the mixture was concentrated to dryness on a rotovap and then under high vacuum to give Intermediate 5b: isopropyl 2′-(chlorocarbonyl)-[1,1′-biphenyl]-2-carboxylate (372 mg, 100% yield).

### Intermediates 6:

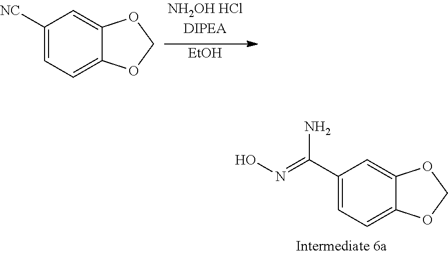 [SMILES: "EtOH" ; N#Cc1ccc2c(c1)OCO2 ; N/C(=N\O)c1ccc2c(c1)OCO2]

DIPEA (12.17 ml, 69.7 mmol) was added to a suspension of piperonylonitrile (5 g, 34.0 mmol) and hydroxylamine hydrochloride (4.72 g, 68.0 mmol) in EtOH (75 mL). After stirring at 20° C. for 21.5 h, the reaction mixture was concentrated to dryness. The resulting colorless oil was cooled to 0° C. and cold water was added (75.0 mL) and the mixture was stirred for 30 minutes to give a white slurry. The solids were filtered, washed with water (2×15 mL) and the resulting solid dried at 40° C. under high vacuum until constant weight to afford Intermediate 6a (Z)—N′-hydroxybenzo[d][1,3]dioxole-5-carboximidamide (5.76 g, 94% yield) as a white solid: ¹H NMR (DMSO-d₆) is consistent with the desired product; MS m/z 181.1 (MH+); HPLC 100%, Rt=0.16 (0.31) min. Alternatively DIPEA can be replaced by sodium carbonate or commercial sodium ethoxide while working in ethanol as solvent for the reaction. Except when commercially available, all N′-hydroxycarboximidamides cited in the examples hereafter were prepared following the recipe outline before for intermediate 6a with the appropriate nitrile precursor and including its variation on the nature of the base used.

### Preparation of Example 1

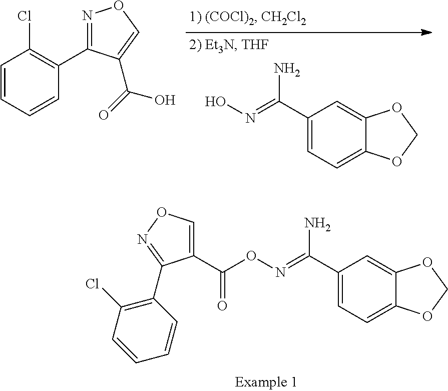 [SMILES: O=C(O)c1conc1-c1ccccc1Cl ; N/C(=N\O)c1ccc2c(c1)OCO2 ; N/C(=N\OC(=O)c1conc1-c1ccccc1Cl)c1ccc2c(c1)OCO2]

Oxalyl chloride (0.196 mL, 2.236 mmol) and DMF (9.70 μl, 0.125 mmol) were added dropwise to a solution of 3-(2-chlorophenyl)isoxazole-4-carboxylic acid (0.200 g, 0.894 mmol) in DCM (3.00 mL) to give an orange solution. Gas evolution was observed and stirring of the resulting yellow solution was maintained at 20° C. for 1.5 h. The mixture was then concentrated to dryness under high vacuum to give an orange oil. Then, a solution of N′-hydroxybenzo[d][1,3]dioxole-5-carboximidamide (0.161 g, 0.894 mmol) in THF (2.5 mL) were added followed by triethylamine (0.150 mL, 1.073 mmol) and the resulting orange slurry was stirred at 20° C. for 5 h. The reaction mixture was then diluted with EtOAc (20 mL) and the organic mixture washed with sat. NaHCO₃ (20 mL) and then with brine (10 mL). The organic layer was dried over anh. MgSO₄, filtered and concentrated (thereafter referred to as the standard or usual isolation procedure in the additional examples described below) to give an orange solid which was slurried overnight in a 1:2 mixture of DCM-Et₂O (4.5 mL). The solid was then filtered, washed with Et₂O (2×1 mL) and dried at 40° C. under high vacuum until constant weight to afford (Z)—N′-((3-(2-chlorophenyl)isoxazole-4-carbonyl)oxy)benzo[d][1,3]dioxole-5-carboximidamide, Example 1 (267 mg, 77% yield) as a white solid: ¹H NMR (400 MHz, DMSO-d₆)_ppm 6.08 (s, 2H) 6.74 (br. s., 2H) 6.99 (d, J=7.83 Hz, 1H) 7.20 (d, J=1.57 Hz, 1H) 7.25 (dd, J=7.80, 1.60 Hz, 1H) 7.46-7.53 (m, 1H) 7.53-7.61 (m, 2H) 7.62-7.67 (m, 1H) 10.09 (s, 1H); MS m/z 386.0530 (MH⁺); HPLC 100%, Rt=1.86 min, column 2.

### Example 2

Using similar procedures as outlined above, a reaction between 2,6-dimethylbenzoyl chloride (Intermediate 5a) and (Z)-2-(2,4-dimethoxyphenyl)-N′-hydroxyacetimidamide followed by flash chromatography of the crude product afforded (Z)-2-(2,4-dimethoxyphenyl)-N′-((2,6-dimethylbenzoyl)oxy)acetimidamide as a solid (100 mg, 75% yield): ¹H NMR (400 MHz, DMSO-d₆)_ppm 2.24 (s, 6H) 3.31 (s, 2H) 3.75 (s, 3H) 3.79 (s, 3H) 6.21 (br. s., 2H) 6.50 (dd, J=8.41, 2.54 Hz, 1H) 6.56 (d, J=2.35 Hz, 1H) 7.081 (d, J=7.70 Hz, 1H) 7.083 (d, J=8.20 Hz, 1H) 7.14 (d, J=8.22 Hz, 1H) 7.23 (dd, J=7.83, 7.50 Hz, 1H); MS m/z 343.1624 (MH⁺); HPLC >99.5%, Rt=1.93 min, column #2.

### Example 3

Following the procedures described above, reaction of 2,6-dimethylbenzoyl chloride (112 mg) and (Z)—N′-hydroxy-2-methoxybenzimidamide (110 mg) gave a solid residue that was swished in Et₂O-pentane with a little DCM to give 102 mg (52%) of (Z)—N′-((2,6-dimethylbenzoyl)oxy)-2-methoxybenzimidamide (Example 3) as a white solid: ¹H NMR (400 MHz, DMSO-d₆)_ppm 2.30 (s, 6H) 3.81 (s, 3H) 6.62 (s, 2H) 6.94-7.02 (m, 1H) 7.05-7.16 (m, 3H) 7.21-7.27 (m, 1H) 7.37 (dd, J=7.43, 1.57 Hz, 1H) 7.41-7.48 (m, 1H); HPLC col #2, >99%, Rt=1.802; MS m/z 299.1411 (MH⁺).

### Example 4

Using the procedures described above, 2,6-bis(trifluoromethyl)benzoyl chloride (60 mg) and (Z)—N′-hydroxybenzo[d][1,3]dioxole-5-carboximidamide (38.1 mg) gave a crude product which was purified by flash chromatography to give after swish in Et₂O-pentane, 53 mg (89%) of example 4, (Z)—N′-((2,6-bis(trifluoromethyl)benzoyl)oxy)benzo[d][1,3]dioxole-5-carboximidamide as a white solid: ¹H NMR (400 MHz, DMSO-d₆)_ppm 6.07 (s, 2H) 6.82 (br. s., 2H) 6.96 (d, J=8.22 Hz, 1H) 7.05-7.35 (br. s., 2H) 7.92-8.02 (m, 1H) 8.22 (d, J=8.22 Hz, 2H); HPLC column #2, >99.5%, Rt 1.83 min; MS m/z 421.0647 (MH⁺).

### Example 5

Following the reaction described above, [1,1′-biphenyl]-2-carbonyl chloride (75 mg) and (Z)—N′-hydroxy-2-(4-methoxyphenyl)acetimidamide 2,2,2-trifluoroacetate (112 mg) were allowed to react in THF to afford a crude product purified by flash chromatography followed by crystallisation in Et₂O and a touch of EtOAc to give 105 mg of example 5, (Z)—N′-(([1,1′-biphenyl]-2-carbonyl)oxy)-2-(4-methoxyphenyl)acetimidamide as white needles: ¹H NMR (400 MHz, DMSO-d₆)_ppm 3.23 (s, 2H) 3.72 (s, 3H) 5.2-6.4 (br. s, 2H) 6.86 (m, J=8.61 Hz, 2H) 7.18 (m, J=8.61 Hz, 2H) 7.32-7.44 (m, 6H) 7.46-7.51 (m, 1H) 7.61 (td, J=7.63, 1.57 Hz, 1H) 7.80 (dd, J=7.63, 0.98 Hz, 1H); HPLC column 2, >99.5%, Rt 2.01; MS m/z 361.1443 (MH⁺).

### Example 6

A crude preparation of 2-methyl-2-phenylpropanoyl chloride (from the corresponding commercial acid and oxalyl chloride as described above) was dissolved in THF (2.5 ml) and (Z)—N′-hydroxybenzo[d][1,3]dioxole-5-carboximidamide (90 mg, 0.499 mmol) was added followed by triethylamine (0.139 ml, 0.999 mmol). When the reaction was completed, the crude product was isolated as usual and purified by Flash chromatography to afford (Z)—N′-((2-methyl-2-phenylpropanoyl)oxy)benzo[d][1,3]dioxole-5-carboximidamide (example 6) as a white powder. ¹H NMR (400 MHz, DMSO-d₆)_ppm 1.62 (s, 6H) 6.07 (s, 2H) 6.31 (br. s., 2H) 6.97 (d, J=7.83 Hz, 1H) 7.18 (d, J=1.96 Hz, 1H) 7.20-7.29 (m, 2H) 7.33-7.39 (m, 2H) 7.41-7.47 (m, 2H); HPLC: >99.5% column 2, Rt 1.89 min; MS m/z 327.1550 (MH⁺).

### Example 7

Following a typical procedure as described above, 2,6-dimethylbenzoyl chloride (70 mg, 0.415 mmol) and (Z)—N′-hydroxy-2-(2-methoxyphenyl)acetimidamide (59.8 mg, 0.332 mmol) were allowed to react in THF (2 ml) in the presence of DIPEA (0.436 mmol). After the usual isolation procedure, the crude solid residue was swished in Et₂O-pentane with a little DCM to give 35 mg (27%) of Example 7, (Z)—N′-((2,6-dimethylbenzoyl)oxy)-2-(2-methoxyphenyl)acetimidamide, as a white solid: ¹H NMR (400 MHz, DMSO-d₆)_ppm 2.25 (s, 6H) 3.40 (s, 2H) 3.81 (s, 3H) 6.28 (br. s., 2H) 6.89-6.95 (m, 1H) 7.00 (dd, J=8.80, 0.98 Hz, 1H) 7.05-7.12 (m, 2H) 7.19-7.28 (m, 3H); HPLC >99%, column #2, Rt 1.901; MS m/z 313.1564 (MH⁺).

### Example 8

Following a reaction procedure described above, 2,6-dimethylbenzoyl chloride (prepared in situ from the corresponding acid (26 mg, 0.173 mmol)) was allowed to react with (Z)-3-(4-bromophenyl)-N′-hydroxypropanimidamide (38.3 mg, 0.157 mmol) in the presence of triethylamine (0.031 ml, 0.220 mmol). Following the usual isolation procedure and purification by Flash chromatography and treatment in Et₂O-pentane, 14 mg (24%) of example 8, (Z)-3-(4-bromophenyl)-N′-((2,6-dimethylbenzoyl)oxy)propanimidamide, were isolated as a solid: ¹H NMR (400 MHz, DMSO-d₆)_ppm 2.23 (s, 6H) 2.31-2.38 (m, 2H) 2.81-2.88 (m, 2H) 6.42 (br. s., 2H) 7.08 (m, J=7.43 Hz, 2H) 7.18-7.27 (m, 3H) 7.43-7.51 (m, 2H); HPLC >99.5%, column #2, Rt=2.054 min; MS m/z 375.0711 (MH⁺).

### Example 9

Following a procedure described above the crude 2,2-diphenylpropanoyl chloride prepared from the commercial acid (113 mg, 0.499 mmol) was allowed to react with (Z)—N′-hydroxybenzo[d][1,3]dioxole-5-carboximidamide (90 mg, 0.499 mmol) in THF in the presence of triethylamine (0.139 mL, 0.999 mmol). After isolation and purification by flash chromatography followed by treatment with Et₂O-pentane, 118 mg (61%) of (Z)—N′-((2,2-diphenylpropanoyl)oxy)benzo[d][1,3]dioxole-5-carboximidamide (example 9) were obtained as a white powder: ¹H NMR (400 MHz, DMSO-d₆)_ppm 2.00 (s, 3H) 6.08 (s, 2H) 6.13 (br. s., 2H) 6.98 (d, J=8.22 Hz, 1H) 7.17-7.32 (m, 8H) 7.33-7.40 (m, 4H); HPLC >99.5%, column 2, Rt 2.086 min; MS m/z 389.1525 (MH⁺).

### Example 10

Following the standard procedure described above, (Z)-3-(4-bromophenyl)-N′-hydroxypropanimidamide (28.5 mg, 0.117 mmol) was allowed to react with commercially available 3-(2-chlorophenyl)-5-methylisoxazole-4-carbonyl chloride (30 mg, 0.117 mmol) in DCM in the presence of triethylamine (0.023 mL, 0.164 mmol) for 3 h. After the usual isolation procedure the crude white solid was swished in Et₂O-pentane with a trace of DCM to afford after drying under high vacuum 38 mg (70%) of example 10, (Z)-3-(4-bromophenyl)-N′-((3-(2-chlorophenyl)-5-methylisoxazole-4-carbonyl)oxy)propanimidamide as a white powder: ¹H NMR (400 MHz, DMSO-d₆)_ppm 2.21-2.31 (m, 2H) 2.69-2.85 (m, 2H) 2.75 (s, 3H) 6.41 (s, 2H) 7.14-7.20 (m, 2H) 7.43-7.48 (m, 2H) 7.48-7.61 (m, 3H) 7.62-7.66 (m, 1H); HPLC >99.5%, column 2, Rt=2.130 min; MS m/z 462.0210 and 464.0191 (MH⁺).

### Example 11

Oxalyl chloride (0.166 mL, 1.896 mmol) and DMF (8.22 μl, 0.106 mmol) were added to a suspension of 3-(2-chlorophenyl)-5-cyclopropylisoxazole-4-carboxylic acid (0.2 g, 0.759 mmol) in DCM (2.54 mL). Gas evolution was observed and stirring of the resulting colorless solution was maintained at 20° C. for 2 hours then the mixture was concentrated to dryness on rotovap then under high vacuum to give a light yellow mixture of oil and solid. Then a solution of N′-hydroxybenzo[d][1,3]dioxole-5-carboximidamide (0.137 g, 0.759 mmol) in THF (2.120 mL, 25.9 mmol) and triethylamine (0.127 mL, 0.910 mmol) were added and the resulting orange slurry was stirred at 20° for 17 h. Following the usual isolation procedure the solid was slurried in DCM (2 mL) and Et₂O (4 mL), filtered, washed with Et₂O (2×1 mL) and dried at 40° C. under high vacuum until constant weight to afford (Z)—N′-((3-(2-chlorophenyl)-5-cyclopropyl isoxazole-4-carbonyl)oxy)benzo[d][1,3]dioxole-5-carboximidamide (276 mg, 0.648 mmol, 85% yield) as a white solid: ¹H NMR (400 MHz, DMSO-d₆)_ppm 1.24-1.30 (m, 2H) 1.30-1.37 (m, 2H) 2.82-2.94 (m, 1H) 5.84 (br. s., 2H) 6.07 (s, 2H) 6.96 (d, J=8.22 Hz, 1H) 7.14 (d, J=1.56 Hz, 1H) 7.19 (dd, J=8.22, 1.56 Hz, 1H) 7.48-7.54 (m, 1H) 7.55-7.63 (m, 2H) 7.63-7.67 (m, 1H); MS m/z 426.0889 (MH⁺); HPLC 100%, Rt=5.78 min, column 2.

### Example 12

Triethylamine (0.094 mL, 0.676 mmol) was added to a mixture of (Z)-4-((4-chlorobenzyl)oxy)-N′-hydroxybenzimidamide (0.156 g, 0.563 mmol) and 3-(2-chlorophenyl)-5-isopropylisoxazole-4-carbonyl chloride (0.160 g, 0.563 mmol) in THF (3.46 mL, 42.2 mmol) Stirring was maintained for 22 h and following the usual isolation procedure gave 290 mg of a white solid which was recrystallized from DCM-Et₂O to afford after filtration and drying at 40° C. under high vacuum until constant weight, 166 mg (56.2% yield) of example 12 (Z)-4-((4-chlorobenzyl)oxy)-N′-((3-(2-chlorophenyl)-5-isopropylisoxazole-4-carbonyl)oxy)benzimidamide as a white solid: ¹H NMR (400 MHz, DMSO-d₆)_ppm 1.38 (d, J=7.04 Hz, 6H) 3.84 (spt, J=7.00 Hz, 1H) 5.15 (s, 2H) 5.81 (br. s., 2H) 7.01-7.08 (m, 2H) 7.42-7.51 (m, 1H) 7.46 (d, J=3.13 Hz, 3H) 7.53 (dd, J=7.43, 1.17 Hz, 1H) 7.55-7.61 (m, 3H) 7.61-7.67 (m, 2H); MS m/z 524.1150 (MH⁺); HPLC 100%, Rt=6.96 min, column 2.

### Example 13

Oxalyl chloride (0.070 mL, 0.797 mmol) and DMF (3.46 μL, 0.045 mmol) were added to a solution of 5-benzyl-3-(2-chlorophenyl)isoxazole-4-carboxylic acid (0.100 g, 0.319 mmol) in DCM (1.1 mL) to give a colorless solution. After gas evolution, stirring was maintained at 20° C. for 2 h to give a yellow solution for 2 h. Concentration to dryness on the rotovap and then under high vacuum gave a mixture of a light yellow oil and solid to which was added a solution of N′-hydroxybenzo[d][1,3]dioxole-5-carboximidamide (0.057 g, 0.319 mmol) and triethylamine (0.053 ml, 0.382 mmol) in THF (0.891 mL). The resulting orange slurry was stirred at 20° C. for 17 h and following the usual isolation procedure described above, the resulting orange foam was purified by flash chromatography and drying at 40° C. under high vacuum until constant weight to give 72 mg (47.5% yield) of example 13 as a white solid: (Z)—N′-((5-benzyl-3-(2-chlorophenyl)isoxazole-4-carbonyl)oxy)benzo[d][1,3]dioxole-5-carboximidamide: ¹H NMR (400 MHz, DMSO-d₆)_ppm 4.61 (s, 2H) 5.91 (br. s., 2H) 6.07 (s, 2H) 6.96 (d, J=8.22 Hz, 1H) 7.15 (d, J=1.96 Hz, 1H) 7.20 (dd, J=8.22, 1.96 Hz, 1H) 7.27-7.34 (m, 1H) 7.35-7.41 (m, 2H) 7.37 (s, 2H) 7.48-7.55 (m, 1H) 7.59 (td, J=7.73, 1.76 Hz, 1H) 7.62-7.68 (m, 2H); MS m/z 476.1042 (MH⁺); HPLC Peak 100%, Rt=6.05 min, column 2.

### Example 14

Triethylamine (0.130 mL, 0.929 mmol) was added to a white suspension of 3-(2-chlorophenyl)-5-methylisoxazole-4-carbonyl chloride (0.200 g, 0.781 mmol) and (Z)—N′-hydroxy-N-methylbenzo[d][1,3]dioxole-5-carboximidamide (0.152 g, 0.781 mmol) in THF (2.5 mL) and the resulting white suspension was stirred at 20° C. for 17 h. Following the usual isolation procedure described above afforded 394 mg of crude product which was further purified by flash chromatography to give 303 mg of a white foam which was crystallized from a mixture of DCM-Et₂O (0.5:2 mL) to give, after drying at 40° C. under high vacuum until constant weight, 205 mg, (63.4% yield) of (Z)—N′-((3-(2-chlorophenyl)-5-methylisoxazole-4-carbonyl)oxy)-N-methylbenzo[d][1,3]dioxole-5-carboximidamide as a white solid: —H NMR (DMSO-d₆) is consistent with desired product although a complex mixture of tautomers or cis/trans were observed; MS m/z 414.0780 (MH+); HPLC 100%, Rt=1.98 min, column 1.

### Example 15

Triethylamine (0.094 mL, 0.676 mmol) was added to a mixture of (Z)—N′-hydroxy-2-(2-methoxyphenyl)acetimidamide (0.107 g, 0.591 mmol) and 3-(2-chlorophenyl)-5-isopropylisoxazole-4-carbonyl chloride (0.160 g, 0.563 mmol) in THF (3.46 mL) to give a yellow solution. After stirring the resulting tan slurry for 22 h, the usual isolation procedure gave a crude solid residue which was recrystallized from a mixture of DCM:ether:hexanes to afford after drying at 40° C. under high vacuum until constant weight, (Z)—N′-((3-(2-chlorophenyl)-5-isopropylisoxazole-4-carbonyl)oxy)-2-(2-methoxyphenyl)acetimidamide (161 mg, 66.8% yield) as a white solid: ¹H NMR (400 MHz, DMSO-d₆)_ppm 1.36 (d, J=7.04 Hz, 6H) 3.29 (s, 2H) 3.79 (spt, J=7.00 Hz, 1H) 3.77 (s, 3H) 4.85 (br. s., 1H) 6.10 (br. s., 1H) 6.89 (td, J=7.43, 1.17 Hz, 1H) 6.96 (dd, J=8.22, 1.17 Hz, 1H) 7.12 (dd, J=7.43, 1.96 Hz, 1H) 7.22 (td, J=7.83, 1.96 Hz, 1H) 7.45-7.52 (m, 1H) 7.57 (qd, J=7.70, 1.56 Hz, 2H) 7.63 (dd, J=7.83, 1.00 Hz, 1H); MS m/z 428.1339 (MH⁺); HPLC 100%, Rt 6.12 min, column 2.

### Example 16

A solution of N′-hydroxybenzo[d][1,3]dioxole-5-carboximidamide (0.093 g, 0.516 mmol) in THF (1.441 mL) and triethylamine (0.086 mL, 0.619 mmol) were added to 3-(2-chlorophenyl)-5-(methoxymethyl)isoxazole-4-carbonyl chloride (prepared from 138 mg (0.516 mmol) of the corresponding acid following the standard procedure described for Intermediate 5a) to give following the usual isolation procedure an orange oil which was crystallized from a mixture of DCM and ether (1:5) which after drying at 50° C. under high vacuum afforded (Z)—N′-((3-(2-chlorophenyl)-5-(methoxymethyl)isoxazole-4-carbonyl)oxy)benzo[d][1,3]dioxole-5-carboximidamide (138 mg, 0.321 mmol, 62.3% yield) as a white solid: ¹H NMR (400 MHz, DMSO-d₆)_ppm 3.42 (s, 3H) 4.96 (s, 2H) 6.08 (s, 2H) 6.26 (br. s., 2H) 6.97 (d, J=8.22 Hz, 1H) 7.17 (d, J=1.57 Hz, 1H) 7.22 (dd, J=8.20, 1.60 Hz, 1H) 7.48-7.55 (m, 1H) 7.56-7.63 (m, 2H) 7.63-7.68 (m, 1H): MS m/z 430.0811 (MH⁺); HPLC 100%, Rt 5.54 min, column 2.

### Example 17

A solution of N′-hydroxybenzo[d][1,3]dioxole-5-carboximidamide (0.120 g, 0.665 mmol) in THF (1.859 mL) and triethylamine (0.185 mL, 1.330 mmol) were added to 3-(2-chlorophenyl)-5-(pyridin-4-yl)isoxazole-4-carbonyl chloride (obtained from the corresponding acid (0.200 g, 0.665 mmol) following the procedure described for intermediate 5a) to give following the usual isolation procedure an orange oil which was crystallized from a mixture of DCM:ether (1:2). After filtration and drying the product at 50° C. under high vacuum until constant weight, 256 mg (0.553 mmol, 83% yield) of (Z)—N′-((3-(2-chlorophenyl)-5-(pyridin-4-yl)isoxazole-4-carbonyl)oxy)benzo[d][1,3]dioxole-5-carboximidamide were isolated as a white solid: ¹H NMR (DMSO-d₆)_{—}6.04 (br. s, 2H) 6.07 (s, 2H) 6.95 (d, J=8.22 Hz, 1H) 7.12 (d, J=1.57 Hz, 1H) 7.18 (dd, J=8.22, 1.57 Hz, 1H) 7.52-7.59 (m, 1H) 7.62 (td, J=7.73, 1.76 Hz, 1H) 7.66-7.74 (m, 2H) 7.94-8.03 (m, 2H) 8.86 (d, J=5.48 Hz, 2H); MS m/z 463.0855 (MH⁺); HPLC 99.8%, Rt 5.76 min, column 2.

### Example 18

Triethylamine (0.112 mL, 0.801 mmol) was added to a freshly prepared solution of 3-(2-chlorophenyl)-5-phenylisoxazole-4-carbonyl chloride (from 0.2 g (0.667 mmol) of the corresponding acid following the procedure outlined in the preparation of Intermediate 5a) and N′-hydroxybenzo[d][1,3]dioxole-5-carboximidamide (0.120 g, 0.667 mmol) in THF (1.9 mL). After stirring for 17 h, the usual isolation procedure as outlined before gave a yellow solid which was recrystallized from DCM-ether (1:3) to give, after drying at 50° C. under high vacuum, 163 mg (52.9% yield) of (Z)—N′-((3-(2-chlorophenyl)-5-phenylisoxazole-4-carbonyl)oxy)benzo[d][1,3]dioxole-5-carboximidamide as a white solid: ¹H NMR (400 MHz, DMSO-d₆) a ppm 5.87 (br. s., 2H) 6.07 (s, 2H) 6.95 (d, J=8.20 Hz, 1H) 7.11 (d, J=1.80 Hz, 1H) 7.17 (dd, J=8.22, 1.80 Hz, 1H) 7.52-7.57 (m, 1H) 7.58-7.69 (m, 5H) 7.71 (dd, J=7.24, 1.76 Hz, 1H) 7.98-8.04 (m, 2H); MS m/z 462.0885 (MH⁺); HPLC 100%, Rt 6.03 min, column #2.

### Example 19

Triethylamine (0.130 mL, 0.929 mmol) was added to a mixture of 3-(2-chlorophenyl)-5-methylisoxazole-4-carbonyl chloride (0.200 g, 0.781 mmol) and (Z)—N′-hydroxy-N,N-dimethylbenzo[d][1,3]dioxole-5-carboximidamide (0.163 g, 0.781 mmol) in THF (2.5 mL) to give a white suspension. After stirring at 20° C. for 20.5 h, the usual isolation procedure gave 333 mg of a white foam which was crystallized from a mixture of DCM-ether-hexanes to give, after drying at 40° C. under high vacuum, 251 mg (75% yield) of (Z)—N′-((3-(2-chlorophenyl)-5-methylisoxazole-4-carbonyl)oxy)-N,N-dimethylbenzo[d][1,3]dioxole-5-carboximidamide as a white solid: ¹H NMR (400 MHz, DMSO-d₆) ∂ ppm 2.29 (s, 3H) 2.72 (s, 6H) 6.10 (s, 2H) 6.53 (dd, J=7.80, 1.60 Hz, 1H) 6.61 (d, J=1.57 Hz, 1H) 6.94 (d, J=7.83 Hz, 1H) 7.27-7.33 (m, 1H) 7.37-7.44 (m, 1H) 7.48-7.57 (m, 2H); MS m/z 428.1008 (MH⁺); HPLC 96.9%, Rt 2.03 min, column 2.

### Example 20

Triethylamine (0.130 mL, 0.929 mmol) was added to a mixture of 3-(2-chlorophenyl)-5-methylisoxazole-4-carbonyl chloride (0.200 g, 0.781 mmol) and (Z)—N-butyl-N′-hydroxybenzo[d][1,3]dioxole-5-carboximidamide (0.213 g, 0.781 mmol) in THF (2.5 mL). After stirring the resulting tan suspension at 20° C. for 18.5 h, the standard isolation procedure gave a residue which was purified by flash chromatography to give 300 mg of a white foam which was crystallized from a 1:2 mixture of DCM-ether to give, after drying at 40° C. under high vacuum, 61 mg (17.13% yield) of (Z)—N-butyl-N′-((3-(2-chlorophenyl)-5-methylisoxazole-4-carbonyl)oxy)benzo[d][1,3]dioxole-5-carboximidamide as a white solid: ¹H NMR (400 MHz, DMSO-d₆) ∂ ppm 0.74 (t, J=7.20 Hz, 3H) 1.02-1.13 (m, 2H) 1.13-1.23 (m, 2H) 2.74-2.85 (m, 2H) 2.79 (s, 3H) 5.39 (t, J=6.26 Hz, 1H) 6.08 (s, 2H) 6.89 (dd, J=7.83, 1.56 Hz, 1H) 6.93 (d, J=1.60 Hz, 1H) 6.98 (d, J=7.80 Hz, 1H) 7.50-7.55 (m, 1H) 7.57-7.63 (m, 2H) 7.65-7.69 (m, 1H); MS m/z 456.1220 (MH⁺); HPLC 100%, Rt 2.20 min, column 2.

### Example 21

Triethylamine (0.130 mL, 0.929 mmol) was added to a suspension of 3-(2-chlorophenyl)-5-methylisoxazole-4-carbonyl chloride (0.200 g, 0.781 mmol) and (Z)—N′-hydroxy-N-(pyridin-3-ylmethyl)benzo[d][1,3]dioxole-5-carboximidamide (0.224 g, 0.781 mmol) in THF (2.5 mL). After stirring at 20° C. overnight and following the usual isolation procedure, gave 384 mg of a crude product which was purified by flash chromatography to afford 331 mg of a white foam. Crystallization from DCM-ether (5 mL, 1:1) collection and drying at 40° C. under high vacuum gave (Z)—N′-((3-(2-chlorophenyl)-5-methyl isoxazole-4-carbonyl)oxy)-N-(pyridin-3-ylmethyl)benzo[d][1,3]dioxole-5-carboximidamide (216 mg, 56.3% yield) as a white solid: ¹H NMR (400 MHz, DMSO-d₆)_ppm 2.77 (s, 3H) 4.11 (d, J=6.65 Hz, 2H) 6.06 (s, 2H) 6.29 (t, J=6.70 Hz, 1H) 6.83 (dd, J=8.02, 1.76 Hz, 1H) 6.90 (d, J=1.80 Hz, 1H) 6.93 (d, J=8.00 Hz, 1H) 7.29-7.36 (m, 1H) 7.38-7.45 (m, 2H) 7.49 (td, J=7.63, 1.96 Hz, 1H) 7.52-7.57 (m, 2H) 8.21 (d, J=1.56 Hz, 1H) 8.44 (dd, J=4.89, 1.76 Hz, 1H); MS m/z 491.1107 (MH⁺); HPLC 100% Rt 1.77 min, column 2.

### Example 22

Solid 3-(2-chlorophenyl)-5-methylisoxazole-4-carbonyl chloride (200 mg, 0.781 mmol) was added followed by diisopropylethylamine (0.239 mL, 1.367 mmol) to a solution of (Z)—N′-hydroxy-2-(2-methoxyphenyl)acetimidamide (246 mg, 1.367 mmol) in THF (5 mL) and DCM (1 mL). After stirring the mixture overnight at room temperature, the usual isolation procedure left an oily residue that was purified by flash chromatography to give 150 mg (48%) of a colorless glass-like gum which was crystallized from Et₂O/EtOAc/Hex to give 145 mg (46%) of (Z)—N′-((3-(2-chlorophenyl)-5-methyl isoxazole-4-carbonyl)oxy)-2-(4-methoxyphenyl)acetimidamide as a white solid: ¹H NMR (400 MHz, DMSO-d₆)_ppm 2.76 (s, 3H) 3.31 (s, 2H) 3.77 (s, 3H) 4.9-6.7 (br. s, 2H) 6.84-6.93 (m, 1H) 6.97 (dd, J=8.41, 0.98 Hz, 1H) 7.15 (dd, J=7.63, 1.76 Hz, 1H) 7.22 (td, J=7.83, 1.57 Hz, 1H) 7.45-7.51 (m, 1H) 7.52-7.59 (m, 2H) 7.59-7.66 (m, 1H); HPLC >99%, Rt 1.98 min, column 2; MS m/z 400.1049 (MH⁺).

### Example 23

Solid 3-(2-chlorophenyl)-5-methylisoxazole-4-carbonyl chloride (200 mg, 0.781 mmol) was added portionwise to a solution of (Z)—N′-hydroxy-2-(4-methoxyphenyl)acetimidamide 2,2,2-trifluoroacetate (230 mg, 0.781 mmol) in THF (5 ml) followed by the addition of diisopropylethylamine (0.300 mL, 1.718 mmol) resulting in a clear solution which was stirred at room temperature overnight. Concentration to remove THF followed by the isolation procedure as described above left a residue which was dissolved in a minimum amount of DCM and further diluted with a mixture of EtOAc-Et₂O-Hexanes to give 230 mg (73%) of (Z)—N′-((3-(2-chlorophenyl)-5-methyl isoxazole-4-carbonyl)oxy)-2-(4-methoxyphenyl)acetimidamide as a white powder: ¹H NMR (400 MHz, DMSO-d₆)_ppm 2.75 (s, 3H) 3.23 (s, 2H) 3.71 (s, 3H) 5.06 (br. s., 1H) 6.25 (br. s., 1H) 6.81-6.88 (m, 2H) 7.13-7.22 (m, 2H) 7.45-7.51 (m, 1H) 7.51-7.59 (m, 2H) 7.59-7.65 (m, 1H); MS m/400.0978 (MH⁺); HPLC 99%; Rt 1.949 min, column 2.

### Example 24

To a solution of (Z)-2-(benzo[d]thiazol-2-yl)-N′-hydroxyacetimidamide (127 mg, 0.615 mmol) in THF (5 mL) was added 3-(2-chlorophenyl)-5-methylisoxazole-4-carbonyl chloride (150 mg, 0.586 mmol) followed by diisopropylethylamine (0.107 mL, 0.615 mmol). The resulting clear yellow solution was stirred at room temperature for 20 h. After isolation following the procedure described above, purification by flash chromatography of the crude product gave 205 mg (82%) of a light yellow foam for (Z)-2-(benzo[d]thiazol-2-yl)-N′-((3-(2-chlorophenyl)-5-methylisoxazole-4-carbonyl)oxy)acetimidamide: ¹H NMR (400 MHz, DMSO-d₆)_ppm 2.77 (s, 3H) 3.93 (s, 2H) 5.54 (br. s., 1H) 6.60 (br. s., 1H) 7.42 (ddd, J=8.02, 7.04, 1.37 Hz, 1H) 7.45-7.52 (m, 2H) 7.53-7.59 (m, 2H) 7.60-7.65 (m, 1H) 7.92-7.97 (m, 1H) 8.07 (dt, J=7.53, 0.93 Hz, 1H); MS m/z 427.0628 (MH⁺); HPLC: >99%, column 2.

### Example 25

N,N-Diisopropylethylamine (0.11 mL, 0.625 mmol) was added to a mixture of 2,5-dimethyl-3-furoyl chloride and (Z)—N′-hydroxy-4-methylbenzimidamide (75 mg, 0.5 mmol) in 3 mL of THF. After 18 h of stirring at room temperature and following the usual isolation procedure, the solid residue was purified by flash chromatography to give 120 mg (88% yield) of (Z)—N′-((2,5-dimethylfuran-3-carbonyl)oxy)-4-methylbenzimidamide as beige solid: ¹H NMR (MeOD):_ppm 2.29 (s, 3H), 2.41 (s, 3H), 2.59 (s, 3H), 6.5 (s, 1H), 7.3 (d, 2H), 7.68 (d, 2H); MS m/z 273.16 (MH⁺); HPLC>99%.

### Example 26

To a solution of 2,4,6-trimethylbenzoyl chloride (obtained as described in Intermediate 5a) in THF (2.5 mL) and (Z)—N′-hydroxybenzo[d][1,3]dioxole-5-carboximidamide (90 mg, 0.499 mmol) was added triethylamine (0.139 mL, 0.999 mmol). After stirring at room temperature for 4 h and following the standard isolation procedure gave a crude product which treated briefly in a mixture of DCM:Et₂O:pentane to afford 124 mg (76%) of (Z)—N′-((2,4,6-rimethylbenzoyl)oxy)benzo[d][1,3]dioxole-5-carboximidamide as a white solid: ¹H NMR (400 MHz, DMSO-d₆)_ppm 2.25 (s, 6H) 2.26 (s, 3H) 6.09 (s, 2H) 6.68 (br. s., 2H) 6.93 (s, 2H) 6.98 (d, J=8.22 Hz, 1H) 7.24 (d, J=1.57 Hz, 1H) 7.29 (dd, J=8.22, 1.96 Hz, 1H); MS m/z 327.1399 (MH⁺); HPLC >99.5%, column 2.

### Example 27

Diisopropylethylamine (0.145 mL, 0.831 mmol) was added to a turbid mixture of [1,1′-biphenyl]-2-carbonyl chloride (75 mg, 0.346 mmol) and (Z)—N′-hydroxy-2-(4-methoxyphenyl)acetimidamide 2,2,2-trifluoroacetate (112 mg, 0.381 mmol) in THF (3 mL). After stirring overnight at room temperature and following the usual isolation procedure gave a residue which was purified by flash chromatography followed by cristallisation in Et₂O (+some EtOAc) to give 105 mg (Z)—N′-(([1,1′-biphenyl]-2-carbonyl)oxy)-2-(4-methoxyphenyl)acetimidamide as white needles: ¹NMR (400 MHz, DMSO-d₆)_ppm 3.23 (s, 2H) 3.72 (s, 3H) 5.2-6.4 (br. s, 2H) 6.86 (m, J=8.61 Hz, 2H) 7.18 (m, J=8.61 Hz, 2H) 7.32-7.44 (m, 6H) 7.46-7.51 (m, 1H) 7.61 (td, J=7.63, 1.57 Hz, 1H) 7.80 (dd, J=7.63, 0.98 Hz, 1H); HPLC>85%, Rt 1.976 min, column 2; MS m/z 361.1443 (MH⁺).

### Example 28

Triethylamine (0.069 mL, 0.491 mmol) was added to a mixture of (Z)—N′-hydroxy-2-(2-methoxyphenyl)acetimidamide (0.077 g, 0.430 mmol) and isopropyl 2′-(chlorocarbonyl)-[1,1′-biphenyl]-2-carboxylate (0.124 g, 0.410 mmol, prepared following the description for intermediate 5b) in THF (2.5 mL) to give a white suspension. After stirring for 17 h and following the usual isolation procedure described above, one obtained 176 mg of a colorless residue which was purified by flash chromatography to afford (Z)-isopropyl 2′-((((1-amino-2-(2-methoxyphenyl)ethylidene)amino)oxy)carbonyl)-[1,1′-biphenyl]-2-carboxylate (183 mg, 0.410 mmol, 100% yield) as a colorless oil: ¹H NMR (400 MHz, DMSO-d₆)_ppm 0.84 (d, J=5.48 Hz, 3H) 0.94 (d, J=5.48 Hz, 3H) 3.27 (s, 2H) 3.77 (s, 3H) 4.78 (dquin, J=12.28, 6.22, 6.22, 6.22, 6.22 Hz, 1H) 6.05 (br. s., 2H) 6.88 (td, J=7.43, 1.17 Hz, 1H) 6.96 (dd, J=8.22, 1.17 Hz, 1H) 7.06 (dd, J=7.43, 1.57 Hz, 1H) 7.17 (dd, J=7.43, 1.17 Hz, 1H) 7.19-7.28 (m, 2H) 7.49 (tdd, J=7.58, 7.58, 5.18, 1.17 Hz, 2H) 7.53-7.62 (m, 2H) 7.84 (dd, J=7.83, 1.17 Hz, 1H) 8.02 (dd, J=7.83, 1.17 Hz, 1H); MS m/z 447.1918 (MH⁺); HPLC 100%, Rt 6.08 min, column 2.

### Example 29

Triethylamine (0.068 mL, 0.489 mmol) was added to a mixture of (Z)-4-((4-chlorobenzyl)oxy)-N′-hydroxybenzimidamide (0.113 g, 0.407 mmol) and 3-methyl-[1,1′-biphenyl]-2-carbonyl chloride (0.094 g, 0.407 mmol) in THF (2.5 mL) to give a white suspension. After stirring the mixture for 17 h, the usual isolation procedure gave 179 mg of a white solid which was crystallized from DCM-Ether (1:2, 5 mL) to afford, drying the product at 40° C. under high vacuum, 139 mg (72.4% yield) of (Z)-4-((4-chlorobenzyl)oxy)-N′-((3-methyl-[1,1′-biphenyl]-2-carbonyl)oxy)benzimidamide as a white solid: ¹H NMR (400 MHz, DMSO-d₆) ∂ ppm 2.37 (s, 3H) 5.15 (s, 2H) 6.40 (br. s., 2H) 7.00-7.07 (m, 2H) 7.26 (d, J=7.83 Hz, 1H) 7.31-7.50 (m, 11H) 7.56-7.63 (m, 2H); MS m/z 471.1489 (MH⁺); HPLC 100%, Rt 6.77 min, column 2.

### Example 30

Triethylamine (0.120 mL, 0.861 mmol) was added to a mixture of 5-methyl-3-phenylisoxazole-4-carbonyl chloride acid prepared as described for intermediate 5a but from the corresponding carboxylic acid (0.142 g, 0.700 mmol) and (Z)—N′-hydroxy-2-methylthiazole-4-carboximidamide (0.110 g, 0.700 mmol) in THF (2.5 mL). After stirring at 20° C. for 18 h, the usual isolation procedure gave a crude solid which was crystallized from DCM-ether (4 mL). Filtration, washing with ether and drying the product at 40° C. under high vacuum until constant weight afforded (E)-2-methyl-N′-((5-methyl-3-phenylisoxazole-4-carbonyl)oxy)thiazole-4-carboximidamide (196 mg, 0.572 mmol, 82% yield) as a white solid: ¹H NMR (400 MHz, DMSO-d₆)_ppm 2.69 (s, 3H) 2.76 (s, 3H) 6.43 (br. s., 2H) 7.47-7.56 (m, 3H) 7.61-7.67 (m, 2H) 7.92 (d, J=0.78 Hz, 1H); MS m/z 343.0840 (MH⁺); HPLC 100%, Rt 1.91 min, column 1.

### Example 31

N,N-Diisopropylethylamine (0.186 mL, 1.063 mmol) was added to a mixture of 3,5-dimethylisoxazole-4-carboxyl chloride (100 mg, 0.709 mmol, obtained as described in the protocol of Intermediate 5a) and (Z)—N′-hydroxy-4-phenoxybenzimidamide (162 mg, 0.709 mmol) in THF (3.5 mL). After stirring at room temperature for 4 h, the standard isolation procedure afforded a crude product which was first purified by flash chromatography (0 to 3% MeOH/DCM or 25 to 80% EOAc/Hex) and finally by preparative TLC (3% MeOH/DCM) to give 53 mg (21%) of (Z)—N′-((3,5-dimethylisoxazole-4-carbonyl)oxy)-4-phenoxybenzimidamide as a beige solid: ¹H NMR (400 MHz, DMSO-d₆)_ppm 2.42 (s, 3H) 2.68 (s, 3H) 6.81 (br. s., 2H) 7.00-7.14 (m, 4H) 7.21 (t, J=7.43 Hz, 1H) 7.38-7.50 (m, 2H) 7.72-7.81 (m, 2H); MS m/z 352.1316 (MH⁺); HPLC: 99%, Rt 1.507, column 2.

### Example 32

Catalytic DMAP (5.19 mg, 0.043 mmol) was added to a mixture of 1-ethyl-3-(3-dimethylaminopropyl)carbodiimide (102 mg, 0.531 mmol), 3,5-dimethylisoxazole-4-carboxylic acid (60 mg, 0.425 mmol) and (Z)-4-((4-chlorobenzyl)oxy)-N′-hydroxybenzimidamide (118 mg, 0.425 mmol) in DCM (4 mL) and the reaction mixture was stirred at room temperature for 2 h. Concentration of the mixture at reduced pressure and following the previously described isolation procedure gave a crude product which was purified by flash chromatography and then treated with a mixture of ether-pentane to give 87 mg (51%) of (Z)-4-((4-chlorobenzyl)oxy)-N′-((3,5-dimethylisoxazole-4-carbonyl)oxy)benzimidamide as a white solid: ¹HNMR (400 MHz, DMSO-d₆)_ppm 2.41 (s, 3H) 2.67 (s, 3H) 5.18 (s, 2H) 6.73 (br. s., 2H) 7.03-7.12 (m, 2H) 7.41-7.53 (m, 4H) 7.66-7.75 (m, 2H); MS m/z 400.1090 (MH⁺); HPLC >96%, Rt 2.135 min, column 2.

### Example 33

Diisopropylethylamine (0.178 mL, 1.016 mmol) was added to a mixture of 1-naphthoyl chloride (prepared from the corresponding acid (125 mg, 0.726 mmol) as described in Intermediate 5a) and with (Z)—N′-hydroxybenzo[d][1,3]dioxole-5-carboximidamide (131 mg, 0.726 mmol) in THF (3 mL). After stirring the resulting clear solution overnight at room temperature, the usual isolation procedure gave a solid residue that was swished in EtOAc-Et₂O-pentane to give 115 mg (47%) of (Z)—N′-((1-naphthoyl)oxy)benzo[d][1,3]dioxole-5-carboximidamide as a white solid: ¹H NMR (400 MHz, DMSO-d₆)_ppm 6.11 (s, 2H) 6.85 (br. s, 2H) 7.03 (d, J=8.22 Hz, 1H) 7.32 (d, J=1.56 Hz, 1H) 7.37 (dd, J=8.02, 1.76 Hz, 1H) 7.53-7.76 (m, 3H) 7.99-8.08 (m, 1H) 8.20 (d, J=8.22 Hz, 1H) 8.34 (dd, J=7.24, 1.37 Hz, 1H) 8.70 (dd, J=8.80, 0.98 Hz, 1H); MS m/z 335.1000 (MH⁺); HPLC >99%, Rt 1.922, column 2.

### Example 34

N,N-diisopropylethylamine (0.115 mL, 0.661 mmol) was added to a mixture of 3-(2-chlorophenyl)-5-methylisoxazole-4-carbonyl chloride (141 mg, 0.551 mmol) and 3-bromo-N-hydroxybenzimidamide (118 mg, 0.551 mmol) in THF (3.0 mL). The resulting yellow solution was stirred overnight at room temperature and following the usual isolation procedure gave a solid residue which was swished in a mixture of Et₂O-EtOAc-hexanes to afford 220 mg (92%) of (Z)-3-Bromo-N′-((3-(2-chlorophenyl)-5-methylisoxazole-4-carbonyl)oxy)benzimidamide as a white solid: MS m/z 435.9895 (MH⁺).

### Example 35

Triethylamine (0.093 mL, 0.666 mmol) was added to a mixture of (Z)—N′-hydroxybenzo[d][1,3]dioxole-5-carboximidamide (0.100 g, 0.555 mmol) and 2-(trifluoromethyl)benzoyl chloride (0.086 mL, 0.583 mmol) in THF (3.00 mL). After stirring the resulting white slurry at room temperature for 18 h, the usual isolation procedure gave 198 mg of crude product which was purified by flash chromatography to afford 177 mg (91% yield) of (Z)—N′-((2-(trifluoromethyl)benzoyl)oxy)benzo[d][1,3]dioxole-5-carboximidamide as a colorless glass-like solid: ¹H NMR (400 MHz, DMSO-d₆)_ppm 6.10 (s, 2H) 6.78 (br. s., 2H) 7.00 (d, J=7.83 Hz, 1H) 7.25 (d, J=1.96 Hz, 1H) 7.30 (dd, J=7.80, 2.00 Hz, 1H) 7.74-7.85 (m, 2H) 7.88-7.92 (m, 1H) 7.95-8.01 (m, 1H): MS m/z 353.0775 (MH⁺); HPLC 100%, Rt 5.34 min, column 2.

### Example 36

Triethylamine (0.039 mL, 0.281 mmol) was added to a mixture of (Z)—N′-hydroxy-4-(1,2,3-thiadiazol-4-yl)benzimidamide (0.054 g, 0.234 mmol) and 3-(2-chlorophenyl)-5-methylisoxazole-4-carbonyl chloride (0.060 g, 0.234 mmol) in THF (1.44 mL) to give a white suspension which was stirred at room temperature for 1.5 h. Following the standard isolation procedure gave a crude solid was treated in EtOAc (3 mL) for 1 hour. The solids were collected and washed with EtOAc (2×1 mL), dried at 40° C. under high vacuum until constant weight_to afford (Z)—N′-((3-(2-chlorophenyl)-5-methyl isoxazole-4-carbonyl)oxy)-4-(1,2,3-thiadiazol-4-yl)benzimidamide (86 mg, 0.196 mmol, 83% yield) as a white solid: ¹H NMR (400 MHz, DMSO-d₆)_ppm 2.83 (s, 3H) 6.24 (br. s., 2H) 7.49-7.56 (m, 1H) 7.56-7.63 (m, 2H) 7.63-7.69 (m, 1H) 7.81-7.88 (m, 2H) 8.19-8.26 (m, 2H) 9.73 (s, 1H); MS m/z 440.0555 (MH⁺); HPLC 100%, Rt 5.686 min, column 2.

### Example 37

Triethylamine (0.079 mL, 0.569 mmol) was added to a mixture of (Z)-2-(4-((4-fluorobenzyl)oxy)phenyl)-N′-hydroxyacetimidamide (0.130 g, 0.474 mmol) and 2,6-dimethylbenzoyl chloride (0.080 g, 0.474 mmol) in THF (2.92 mL). After stirring the resulting tan slurry for 18.5 h, the standard isolation procedure gave a yellow oil which was purified by flash chromatography to give (Z)—N′-((2,6-dimethylbenzoyl)oxy)-2-(4-((4-fluorobenzyl)oxy)phenyl)acetimidamide (82 mg, 42.5% yield) as a white solid: ¹H NMR (400 MHz, DMSO-d₆)_ppm 2.24 (s, 6H) 3.30 (s, 2H) 5.07 (s, 2H) 6.36 (br. s., 2H) 6.91-6.99 (m, 2H) 7.08 (d, J=7.43 Hz, 2H) 7.17-7.29 (m, 5H) 7.45-7.53 (m, 2H); MS m/z 407.1770 (MH⁺); HPLC 100%, Rt 5.99 min, column 2.

### Example 38

Triethylamine (0.069 mL, 0.491 mmol) was added to a mixture of (Z)—N′-hydroxybenzo[d][1,3]dioxole-5-carboximidamide (0.074 g, 0.410 mmol) and isopropyl 2′-(chlorocarbonyl)-[1,1′-biphenyl]-2-carboxylate (0.124 g, 0.410 mmol, prepared following the description for intermediate 5b) in THF (2.52 mL) to give a white suspension. After stirring for 16.5 h, the isolation procedure gave 221 mg of a colorless oil which was crystallized from ether (8 mL) and after washing with ether, filtration and drying the crystals at 40° C. under high vacuum until one obtained 162 mg (89% yield) of (Z)-isopropyl 2′-((((amino(benzo[d][1,3]dioxol-5-yl)methylene)amino)oxy)carbonyl)-[1,1′-biphenyl]-2-carboxylate as a white solid: ¹H NMR (400 MHz, DMSO-d₆)_ppm 0.81-0.91 (m, 3H) 0.91-0.99 (m, 3H) 4.73-4.86 (m, 1H) 6.07 (s, 2H) 6.19 (br. s., 2H) 6.95 (d, J=8.22 Hz, 1H) 7.15 (d, J=1.57 Hz, 1H) 7.17-7.22 (m, 2H) 7.27 (dd, J=7.63, 0.98 Hz, 1H) 7.46-7.56 (m, 2H) 7.56-7.63 (m, 2H) 7.86 (dd, J=7.83, 1.00 Hz, 1H) 8.12 (dd, J=7.83, 1.17 Hz, 1H); MS m/z 447.1573 (MH⁺); HPLC 100%, Rt 5.91 min, column 2.

### Example 39

Triethylamine (0.109 mL, 0.780 mmol) was added to a mixture of 2,6-dimethylbenzoyl chloride (65.8 mg, 0.390 mmol) and (Z)-2-(2,4-dimethoxyphenyl)-N′-hydroxyacetimidamide (82 mg, 0.390 mmol) in THF (2 mL). After stirring the suspension at room temperature for 1 h, the usual isolation procedure gave a solid residue which was purified by flash chromatography and then treated with a mixture of ether-pentane (with a trace of DCM) to give 100 mg (75%) of (Z)-2-(2,4-dimethoxyphenyl)-N′-((2,6-dimethylbenzoyl)oxy)acetimidamide as solid: ¹H NMR (400 MHz, DMSO-d₆)_ppm 2.24 (s, 6H) 3.31 (s, 2H) 3.75 (s, 3H) 3.79 (s, 3H) 6.21 (br. s., 2H) 6.50 (dd, J=8.41, 2.54 Hz, 1H) 6.56 (d, J=2.35 Hz, 1H) 7.081 (d, J=7.70 Hz, 1H) 7.083 (d, J=8.20 Hz, 1H) 7.14 (d, J=8.22 Hz, 1H) 7.23 (dd, J=7.83, 7.50 Hz, 1H); MS m/z 343.1624 (MH⁺); HPLC >99.5%, Rt 1.902 min, column 2.

### Example 40

Diisopropylethylamine (68 μL, 0.389 mmol) was added to a mixture of crude 2,6-dimethylbenzoyl chloride (60 mg, 0.356 mmol, prepared as described previously for Intermediate 5a) and (Z)-4-((4-chlorobenzyl)oxy)-N′-hydroxybenzimidamide (98 mg, 0.354 mmol) in THF (3 mL). After stirring for 48 h at room temperature, the standard isolation procedure gave a crude residue was swished overnight in ether-pentane to give 135 mg (93%) of (Z)-4-((4-chlorobenzyl)oxy)-N′-((2,6-dimethylbenzoyl)oxy)benzimidamide as a white solid: ¹H NMR (400 MHz, DMSO-d₆)_ppm 2.29 (s, 6H) 5.17 (s, 2H) 6.72 (br. s., 2H) 7.07 (m, J=9.00 Hz, 2H) 7.11 (d, J=7.43 Hz, 2H) 7.22-7.29 (m, 1H) 7.42-7.53 (m, 4H) 7.69 (m, J=9.00 Hz, 2H); MS m/z 409.1329 (MH⁺); HPLC >99%, Rt 2.191 min, column 2.

### Example 41

Triethylamine (0.098 mL, 0.706 mmol) was added to solution of crude 2,6-dimethylbenzoyl chloride (85 mg, 0.504 mmol, prepared from the corresponding acid following the procedure described for Intermediate 5a) and of (Z)-2-((4-fluorobenzyl)oxy)-N′-hydroxybenzimidamide (131 mg, 0.504 mmol) in DCM (3 mL). The reaction was stirred at room temperature for 2 h and the standard isolation procedure gave a crude product which was purified by flash chromatography to give 107 m g (54%) of (Z)—N′-((2,6-dimethylbenzoyl)oxy)-2-((4-fluorobenzyl)oxy)benzimidamide as a white solid: ¹H NMR (400 MHz, DMSO-d₆)_ppm 2.29 (s, 6H) 5.15 (s, 2H) 6.70 (s, 2H) 7.02 (td, J=7.43, 1.17 Hz, 1H) 7.07-7.13 (m, 2H) 7.13-7.21 (m, 3H) 7.21-7.27 (m, 1H) 7.40 (dd, J=7.63, 1.76 Hz, 1H) 7.42-7.48 (m, 1H) 7.52-7.61 (m, 2H); MS m/z 393.1631 (MH⁺); HPLC >99.5%, Rt 2.072 min, column 2.

### Example 42

Triethylamine (0.040 mL, 0.285 mmol) was added to a mixture of (Z)—N′-hydroxy-4-(1,2,3-thiadiazol-4-yl)benzimidamide (0.052 g, 0.237 mmol) and 2,6-dimethylbenzoyl chloride (0.04 g, 0.237 mmol) in THF (1.5 mL) to give a white suspension. After stirring the mixture at room temperature for 18.5 h. the standard isolation protocol gave a white solid which was crystallized from EtOAc (2 mL). Filtration and drying at 40° C. under high vacuum afforded (Z)—N′-((2,6-dimethylbenzoyl)oxy)-4-(1,2,3-thiadiazol-4-yl)benzimidamide (63 mg, 75% yield) as a white solid: ¹H NMR (400 MHz, DMSO-d₆)_ppm 2.32 (s, 6H) 6.94 (br. s., 2H) 7.14 (d, J=7.43 Hz, 2H) 7.24-7.32 (m, 1H) 7.90-7.97 (m, 2H) 8.20-8.28 (m, 2H) 9.75 (s, 1H); MS m/z 353.1062 (MH⁺); HPLC 100%, Rt 5.48 min, column 2.

### Example 43

Triethylamine (0.139 mL, 0.999 mmol) was added to a mixture of 2-methyl-1-naphthoyl chloride (obtained from the corresponding acid (93 mg, 0.499 mmol) following the description offered for Intermediate 5a) and (Z)—N′-hydroxybenzo[d][1,3]dioxole-5-carboximidamide (90 mg, 0.499 mmol) in THF (2.5 mL). The resulting suspension was stirred at room temperature for 4 h and then, following the usual isolation protocol described before, the crude product was treated with a mixture of DCM-Et₂O-pentane to give 135 mg (78%) of (Z)—N′-((2-methyl-1-naphthoyl)oxy)benzo[d][1,3]dioxole-5-carboximidamide as a white powder: ¹H NMR (400 MHz, DMSO-d₆)_ppm 2.50 (s, 3H) 6.10 (s, 2H) 6.74 (br. s., 2H) 7.00 (d, J=8.22 Hz, 1H) 7.25 (d, J=1.57 Hz, 1H) 7.31 (dd, J=8.22, 1.56 Hz, 1H) 7.47 (d, J=8.61 Hz, 1H) 7.50-7.55 (m, 1H) 7.55-7.62 (m, 1H) 7.72-7.79 (m, 1H) 7.97 (dd, J=8.02, 2.93 Hz, 2H); MS m/z 349.1201 (MH⁺); HPLC >99.5%, Rt 1.930 min, column 2.

### Example 44

Diisopropylethylamine (0.173 mL, 0.989 mmol) was added to a mixture of 2-benzylbenzoyl chloride (produced from the corresponding acid (140 mg, 0.660 mmol) following the procedure described for Intermediate 5a) and (Z)—N′-hydroxybenzo[d][1,3]dioxole-5-carboximidamide (119 mg, 0.660 mmol) in THF (2 mL). Stirring was maintained at room temperature overnight and following the usual isolation procedure gave a residue that was swished in EtOAc Et₂O-pentane to give 145 mg (59%) of (Z)—N′-((2-benzylbenzoyl)oxy)benzo[d][1,3]dioxole-5-carboximidamide as a white solid: ¹H NMR (400 MHz, DMSO-d₆)_ppm 4.32 (s, 2H) 6.10 (s, 2H) 6.73 (br. s., 2H) 7.00 (d, J=8.22 Hz, 1H) 7.13-7.20 (m, 3H) 7.22-7.29 (m, 3H) 7.29-7.33 (m, 2H) 7.36 (td, J=7.63, 1.17 Hz, 1H) 7.49-7.54 (m, 1H) 8.00 (dd, J=7.83, 1.17 Hz, 1H); MS m/z 375.1351 (MH⁺); HPLC >97%, Rt 2.05 min, column 2.

### Example 45

Triethylamine (0.081 mL, 0.583 mmol) was added to a mixture of dibenzo[c,e]oxepine-5,7-dione (0.124 g, 0.555 mmol) and (Z)—N′-hydroxybenzo[d][1,3]dioxole-5-carboximidamide (0.100 g, 0.555 mmol) in THF (3.00 mL) to give a colorless solution. After stirring the resulting colorless solution at room temperature for 20 h, the usual isolation procedure gave 300 mg of a white foam which was crystallized from DCM (3 mL) to afford, after drying at 40° C. under high vacuum, 188 mg (84% yield) of (Z)-2′-((((amino(benzo[d][1,3]dioxol-5-yl)methylene)amino)oxy)carbonyl)-[1,1′-biphenyl]-2-carboxylic acid as a white solid: ¹H NMR (400 MHz, DMSO-d₆)_ppm 6.06 (s, 2H) 6.13 (br. s., 2H) 6.95 (d, J=8.22 Hz, 1H) 7.15 (d, J=1.56 Hz, 1H) 7.17-7.25 (m, 3H) 7.43-7.53 (m, 2H) 7.53-7.62 (m, 2H) 7.89 (dd, J=7.83, 1.17 Hz, 1H) 8.08 (dd, J=7.83, 1.17 Hz, 1H) 12.59 (br. s., 1H); MS m/z 405.1090 (MH⁺); HPLC 100%, Rt 5.48 min, column 2.

### Example 46

N,N-Diisopropylethylamine (0.149 mL, 0.854 mmol) was added to a mixture of 3-(2-chlorophenyl)-5-methylisoxazole-4-carbonyl chloride (175 mg, 0.683 mmol) and N-hydroxy-4-methoxybenzimidamide (119 mg, 0.718 mmol) in THF (4 mL). After stirring at room temperature overnight, the usual isolation procedure gave a residue which was swished in MeOH containing a trace of DCM to give 186 mg (71%) of (Z)—N′-((3-(2-chlorophenyl)-5-methylisoxazole-4-carbonyl)oxy)-4-methoxybenzimidamide as a white cotton-like solid: ¹H NMR (400 MHz, DMSO-d₆)_ppm 2.81 (s, 3H) 3.78 (s, 3H) 6.00 (br. s., 1H) 6.92-7.02 (m, 2H) 7.48-7.55 (m, 1H) 7.55-7.68 (m, 4H); MS m/z 386.0903 (MH⁺); HPLC >99%, Rt 1.936 min, column 2.

### Example 47

N,N-Diisopropyyethylamine (0.123 mL, 0.704 mmol) was added to a mixture of N-hydroxyquinoline-2-carboximidamide (120 mg, 0.640 mmol) and 3-(2-chlorophenyl)-5-methylisoxazole-4-carbonyl chloride (164 mg, 0.640 mmol) in THF (2.9 mL). After stirring for 18 h, the standard isolation procedure led to a crude residue which was swished in a mixture of DCM:Et₂O:Hexanes to afford after filtration and air drying, 170 mg (65%) of (Z)—N′-((3-(2-chlorophenyl)-5-methylisoxazole-4-carbonyl)oxy)quinoline-2-carboximidamide as a white solid: ¹H NMR (400 MHz, DMSO-d₆)_ppm 2.85 (s, 3H) 5.68 (s, 1H) 6.98 (s, 1H) 7.50-7.56 (m, 1H) 7.56-7.73 (m, 4H) 7.85 (td, J=7.63, 1.57 Hz, 1H) 8.00 (d, J=8.61 Hz, 1H) 8.10 (d, J=8.22 Hz, 1H) 8.05 (d, J=7.83 Hz, 1H) 8.47 (d, J=8.61 Hz, 1H); MS m/z 407.0815 (MH⁺); HPLC: >95% purity, Rt 2.147 min, column 2.

### Example 48

(Z)—N′-hydroxy-4-(1,2,3-thiadiazol-4-yl)benzimidamide (0.054 g, 0.234 mmol) and 3-(2-chlorophenyl)-5-methylisoxazole-4-carbonyl chloride (0.060 g, 0.234 mmol) were mixed in THF (1.4 mL) followed by the addition of triethylamine (0.039 mL, 0.281 mmol). After stirring for 1.5 h, the standard isolation procedure afforded a white solid which was stirred in 3 mL of EtOAc for 1 h to give, after drying at 40° C. under high vacuum until constant weight, (Z)—N′-((3-(2-chlorophenyl)-5-methylisoxazole-4-carbonyl)oxy)-4-(1,2,3-thiadiazol-4-yl)benzimidamide (86 mg, 0.196 mmol, 83% yield) as a white solid: ¹H NMR (400 MHz, DMSO-d₆)_ppm 2.83 (s, 3H) 6.24 (br. s., 2H) 7.49-7.56 (m, 1H) 7.56-7.63 (m, 2H) 7.63-7.69 (m, 1H) 7.81-7.88 (m, 2H) 8.19-8.26 (m, 2H) 9.73 (s, 1H); MS m/z 440.0555 (MH⁺); HPLC 100%, Rt=5.62 min, column 2.

### Example 49

Diisopropylethylamine (0.078 mL, 0.449 mmol) was added to a suspension of 3-(2-chlorophenyl)-5-methylisoxazole-4-carbonyl chloride (100 mg, 0.390 mmol) and (Z)-4-((6-chloropyridin-3-yl)methoxy)-N′-hydroxybenzimidamide (105 mg, 0.379 mmol) in THF (2 mL). After 15 h stirring at room temperature, the standard isolation procedure led to a solid which was stirred in a mixture of Et₂O:EtOAc to afford 155 mg (80%) of (Z)—N′-((3-(2-chlorophenyl)-5-methylisoxazole-4-carbonyl)oxy)-4-((6-chloropyridin-3-yl)methoxy)benzimidamide as white cotton-like solid: ¹H NMR (400 MHz, DMSO-d₆)_ppm 2.81 (s, 3H) 5.21 (s, 2H) 6.04 (br. s., 2H) 7.08 (d, J=9.00 Hz, 2H) 7.47-7.55 (m, 1H) 7.55-7.71 (m, 5H) 7.95 (dd, J=8.22, 2.35 Hz, 1H) 8.53 (dd, J=2.35, 0.78 Hz, 1H); MS m/z 497.0778 and 499.0754 (MH⁺); HPLC >99%, Rt 2.070 min, column 2.

### Example 50

Solid (Z)—N′-hydroxy-3-methoxybenzimidamide (162 mg, 0.976 mmol) was added to a solution of 3-(2-chlorophenyl)-5-methylisoxazole-4-carbonyl chloride (200 mg, 0.781 mmol) (obtained as described in Intermediate 5a) in THF (4 mL) followed by diisopropylethylamine (0.191 ml, 1.093 mmol). After stirring for 20 h, the standard isolation procedure gave a solid residue that was swished for 48 h in EtOAc:Et₂O:Hex (1:2:1) and then filtered, air dried under high vacuum for 4 h to give 275 mg (91%) of ((Z)—N′-((3-(2-chlorophenyl)-5-methylisoxazole-4-carbonyl)oxy)-3-methoxybenzimidamide as a white solid: ¹H NMR (400 MHz, DMSO-d₆)_ppm 2.81 (s, 3H) 3.78 (s, 3H) 6.14 (br. s., 2H) 7.06 (ddd, J=8.22, 2.54, 0.98 Hz, 1H) 7.16-7.21 (m, 1H) 7.24 (dt, J=7.92, 1.12 Hz, 1H) 7.34 (t, J=7.83 Hz, 1H) 7.52 (td, J=7.34, 1.37 Hz, 1H) 7.55-7.62 (m, 2H) 7.62-7.69 (m, 1H); MS m/z 386.0920 (MH⁺); HPLC >99%, Rt 1.917 min, column 2.

TABLE 1

| Example # | Structure | EC₅₀ (Range) |
|---|---|---|
| 1 | 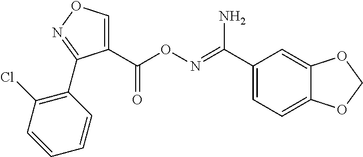 [SMILES: N/C(=N\OC(=O)c1conc1-c1ccccc1Cl)c1ccc2c(c1)OCO2] | B |
| 2 | 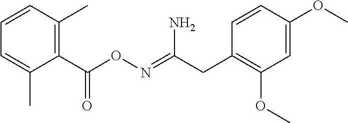 [SMILES: COc1ccc(C/C(N)=N/OC(=O)c2c(C)cccc2C)c(OC)c1] | D |
| 3 | 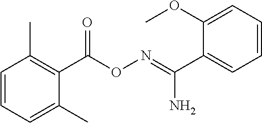 [SMILES: COc1ccccc1/C(N)=N/OC(=O)c1c(C)cccc1C] | D |
| 4 | 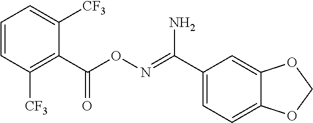 [SMILES: N/C(=N\OC(=O)c1c(C(F)(F)F)cccc1C(F)(F)F)c1ccc2c(c1)OCO2] | B |
| 5 | 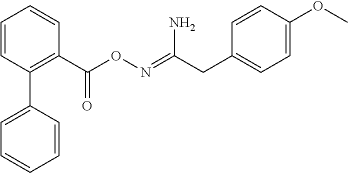 [SMILES: COc1ccc(C/C(N)=N/OC(=O)c2ccccc2-c2ccccc2)cc1] | D |
| 6 | 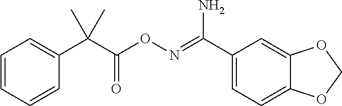 [SMILES: CC(C)(C(=O)O/N=C(\N)c1ccc2c(c1)OCO2)c1ccccc1] | C |
| 7 | 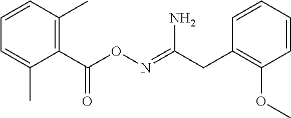 [SMILES: COc1ccccc1C/C(N)=N/OC(=O)c1c(C)cccc1C] | C |
| 8 | 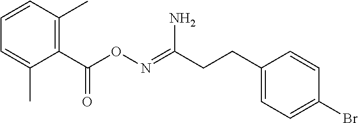 [SMILES: Cc1cccc(C)c1C(=O)O/N=C(\N)CCc1ccc(Br)cc1] | D |
| 9 | 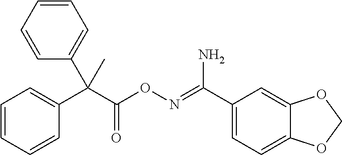 [SMILES: CC(C(=O)O/N=C(\N)c1ccc2c(c1)OCO2)(c1ccccc1)c1ccccc1] | D |
| 10 | 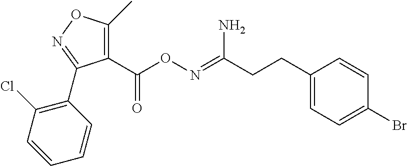 [SMILES: Cc1onc(-c2ccccc2Cl)c1C(=O)O/N=C(\N)CCc1ccc(Br)cc1] | D |
| 11 | 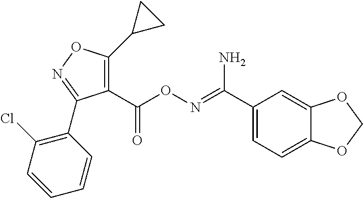 [SMILES: N/C(=N\OC(=O)c1c(-c2ccccc2Cl)noc1C1CC1)c1ccc2c(c1)OCO2] | D |
| 12 | 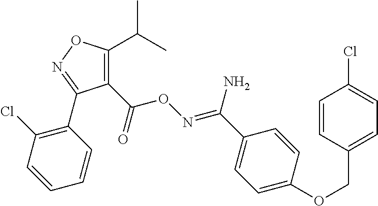 [SMILES: CC(C)c1onc(-c2ccccc2Cl)c1C(=O)O/N=C(\N)c1ccc(OCc2ccc(Cl)cc2)cc1] | C |
| 13 | 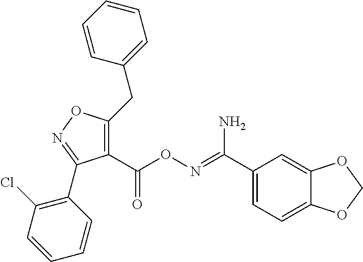 [SMILES: N/C(=N\OC(=O)c1c(-c2ccccc2Cl)noc1Cc1ccccc1)c1ccc2c(c1)OCO2] | D |
| 14 | 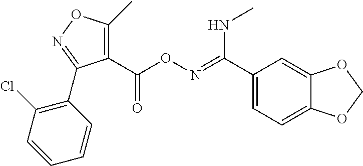 [SMILES: CN/C(=N\OC(=O)c1c(-c2ccccc2Cl)noc1C)c1ccc2c(c1)OCO2] | D |
| 15 | 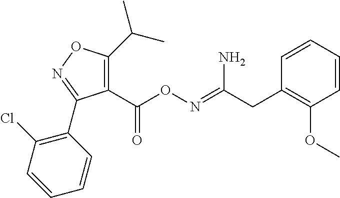 [SMILES: COc1ccccc1C/C(N)=N/OC(=O)c1c(-c2ccccc2Cl)noc1C(C)C] | D |
| 16 | 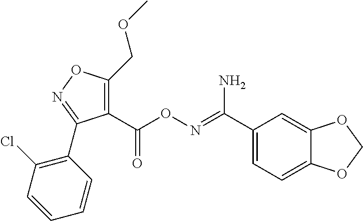 [SMILES: COCc1onc(-c2ccccc2Cl)c1C(=O)O/N=C(\N)c1ccc2c(c1)OCO2] | C |
| 17 | 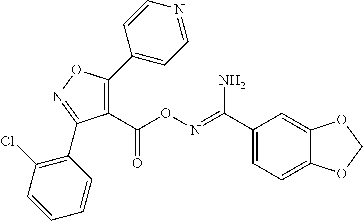 [SMILES: N/C(=N\OC(=O)c1c(-c2ccccc2Cl)noc1-c1ccncc1)c1ccc2c(c1)OCO2] | B |
| 18 | 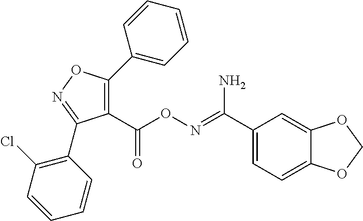 [SMILES: N/C(=N\OC(=O)c1c(-c2ccccc2Cl)noc1-c1ccccc1)c1ccc2c(c1)OCO2] | C |
| 19 | 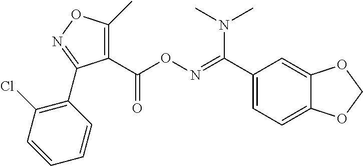 [SMILES: Cc1onc(-c2ccccc2Cl)c1C(=O)O/N=C(/c1ccc2c(c1)OCO2)N(C)C] | B |
| 20 | 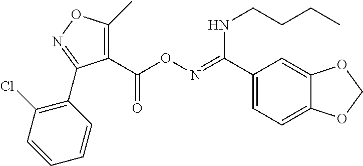 [SMILES: CCCCN/C(=N\OC(=O)c1c(-c2ccccc2Cl)noc1C)c1ccc2c(c1)OCO2] | D |
| 21 | 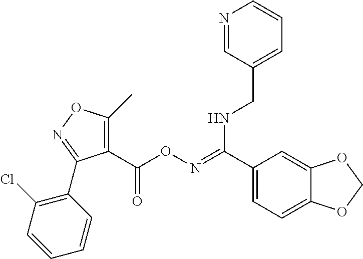 [SMILES: Cc1onc(-c2ccccc2Cl)c1C(=O)O/N=C(\NCc1cccnc1)c1ccc2c(c1)OCO2] | C |
| 22 | 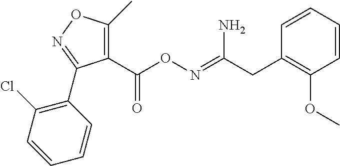 [SMILES: COc1ccccc1C/C(N)=N/OC(=O)c1c(-c2ccccc2Cl)noc1C] | D |
| 23 | 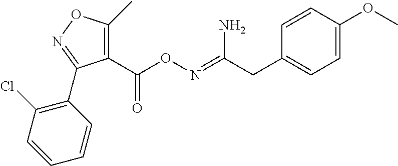 [SMILES: COc1ccc(C/C(N)=N/OC(=O)c2c(-c3ccccc3Cl)noc2C)cc1] | C |
| 24 | 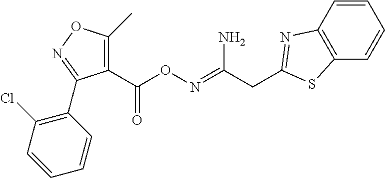 [SMILES: Cc1onc(-c2ccccc2Cl)c1C(=O)O/N=C(\N)Cc1nc2ccccc2s1] | B |
| 25 | 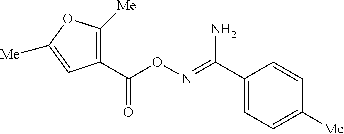 [SMILES: Cc1ccc(/C(N)=N/OC(=O)c2cc(C)oc2C)cc1] | B |
| 26 | 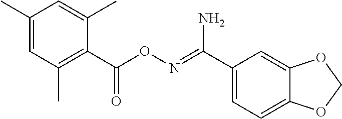 [SMILES: Cc1cc(C)c(C(=O)O/N=C(\N)c2ccc3c(c2)OCO3)c(C)c1] | D |
| 27 | 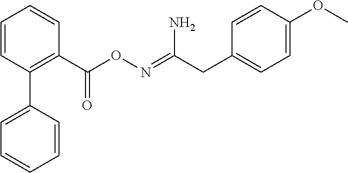 [SMILES: COc1ccc(C/C(N)=N/OC(=O)c2ccccc2-c2ccccc2)cc1] | D |
| 28 | 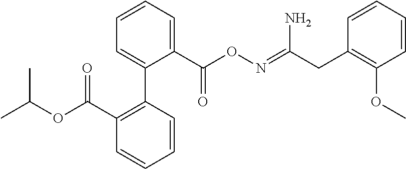 [SMILES: COc1ccccc1C/C(N)=N/OC(=O)c1ccccc1-c1ccccc1C(=O)OC(C)C] | D |
| 29 | 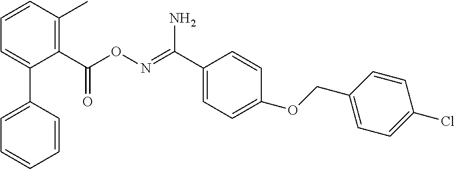 [SMILES: Cc1cccc(-c2ccccc2)c1C(=O)O/N=C(\N)c1ccc(OCc2ccc(Cl)cc2)cc1] | D |
| 30 | 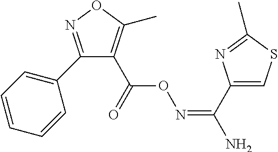 [SMILES: Cc1nc(/C(N)=N\OC(=O)c2c(-c3ccccc3)noc2C)cs1] | B |
| 31 | 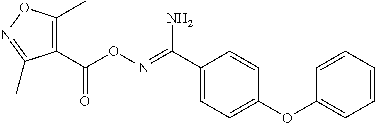 [SMILES: Cc1noc(C)c1C(=O)O/N=C(\N)c1ccc(Oc2ccccc2)cc1] | B |
| 32 | 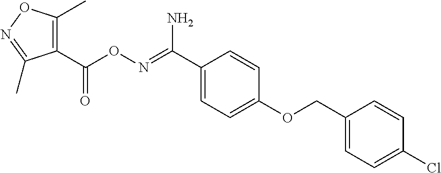 [SMILES: Cc1noc(C)c1C(=O)O/N=C(\N)c1ccc(OCc2ccc(Cl)cc2)cc1] | C |
| 33 | 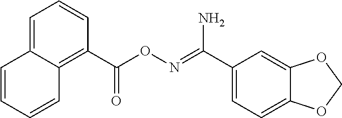 [SMILES: N/C(=N\OC(=O)c1cccc2ccccc12)c1ccc2c(c1)OCO2] | C |
| 34 | 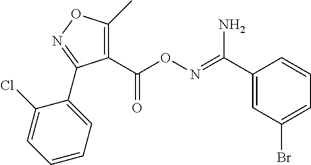 [SMILES: Cc1onc(-c2ccccc2Cl)c1C(=O)O/N=C(\N)c1cccc(Br)c1] | B |
| 35 | 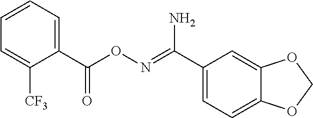 [SMILES: N/C(=N\OC(=O)c1ccccc1C(F)(F)F)c1ccc2c(c1)OCO2] | B |
| 36 | 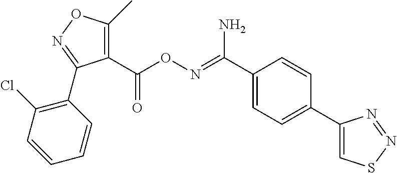 [SMILES: Cc1onc(-c2ccccc2Cl)c1C(=O)O/N=C(\N)c1ccc(-c2csnn2)cc1] | C |
| 37 | 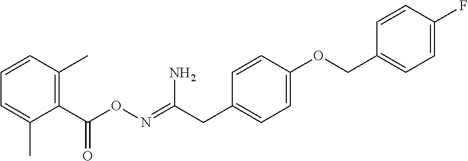 [SMILES: Cc1cccc(C)c1C(=O)O/N=C(\N)Cc1ccc(OCc2ccc(F)cc2)cc1] | C |
| 38 | 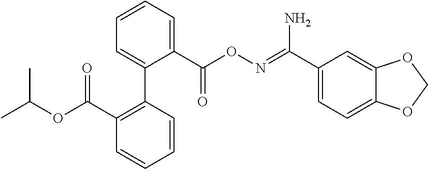 [SMILES: CC(C)OC(=O)c1ccccc1-c1ccccc1C(=O)O/N=C(\N)c1ccc2c(c1)OCO2] | D |
| 39 | 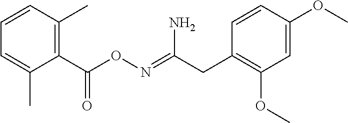 [SMILES: COc1ccc(C/C(N)=N/OC(=O)c2c(C)cccc2C)c(OC)c1] | D |
| 40 | 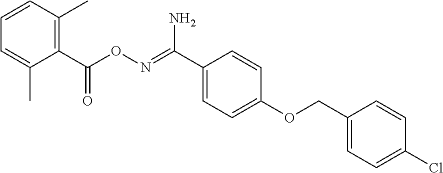 [SMILES: Cc1cccc(C)c1C(=O)O/N=C(\N)c1ccc(OCc2ccc(Cl)cc2)cc1] | D |
| 41 | 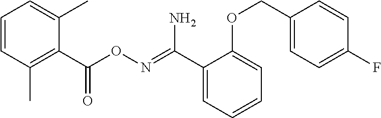 [SMILES: Cc1cccc(C)c1C(=O)O/N=C(\N)c1ccccc1OCc1ccc(F)cc1] | D |
| 42 | 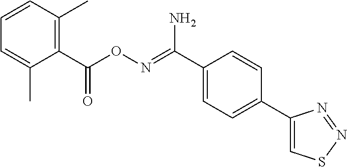 [SMILES: Cc1cccc(C)c1C(=O)O/N=C(\N)c1ccc(-c2csnn2)cc1] | C |
| 43 | 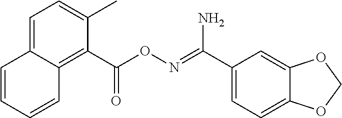 [SMILES: Cc1ccc2ccccc2c1C(=O)O/N=C(\N)c1ccc2c(c1)OCO2] | C |
| 44 | 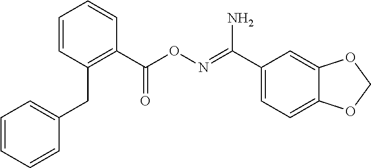 [SMILES: N/C(=N\OC(=O)c1ccccc1Cc1ccccc1)c1ccc2c(c1)OCO2] | C |
| 45 | 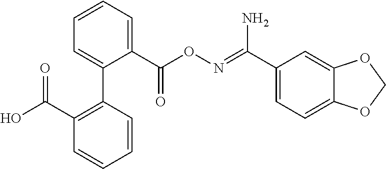 [SMILES: N/C(=N\OC(=O)c1ccccc1-c1ccccc1C(=O)O)c1ccc2c(c1)OCO2] | A |
| 46 | 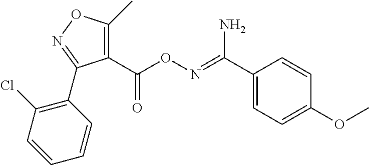 [SMILES: COc1ccc(/C(N)=N/OC(=O)c2c(-c3ccccc3Cl)noc2C)cc1] | C |
| 47 | 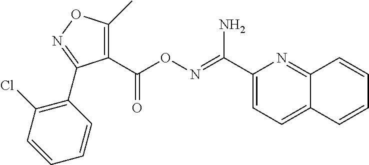 [SMILES: Cc1onc(-c2ccccc2Cl)c1C(=O)O/N=C(\N)c1ccc2ccccc2n1] | B |
| 48 | 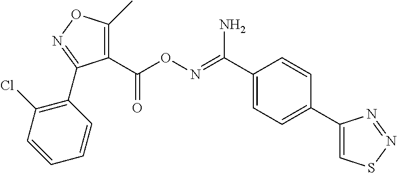 [SMILES: Cc1onc(-c2ccccc2Cl)c1C(=O)O/N=C(\N)c1ccc(-c2csnn2)cc1] | C |
| 49 | 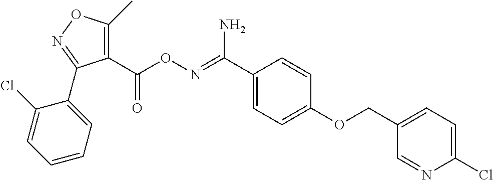 [SMILES: Cc1onc(-c2ccccc2Cl)c1C(=O)O/N=C(\N)c1ccc(OCc2ccc(Cl)nc2)cc1] | D |
| 50 | 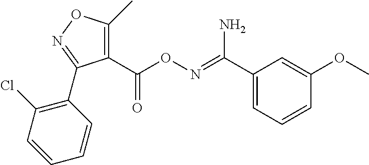 [SMILES: COc1cccc(/C(N)=N/OC(=O)c2c(-c3ccccc3Cl)noc2C)c1] | C |

Table 1: Examples of compounds made and tested. The following denominations were used to report EC₅₀ values: A for >10,000 nM; B for 1,000 to 10,000 nM; C for 500 to 1,000 nM; D for <500 nM.

TABLE 2

| Compound | Structure | EC₅₀ | |
|---|---|---|---|
| 60 | 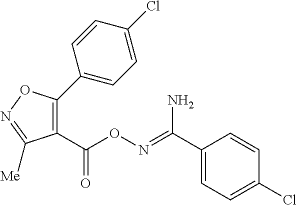 [SMILES: Cc1noc(-c2ccc(Cl)cc2)c1C(=O)O/N=C(\N)c1ccc(Cl)cc1] | C | Maybridge |
| 61 | 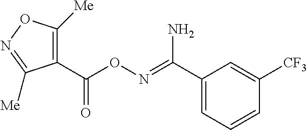 [SMILES: Cc1noc(C)c1C(=O)O/N=C(\N)c1cccc(C(F)(F)F)c1] | B | Maybridge |
| 62 | 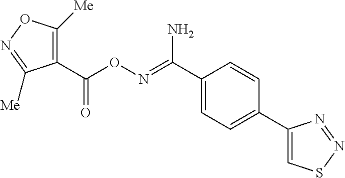 [SMILES: Cc1noc(C)c1C(=O)O/N=C(\N)c1ccc(-c2csnn2)cc1] | C | Maybridge |
| 63 | 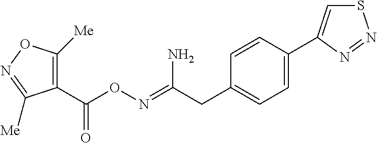 [SMILES: Cc1noc(C)c1C(=O)O/N=C(\N)Cc1ccc(-c2csnn2)cc1] | C | Maybridge |
| 64 | 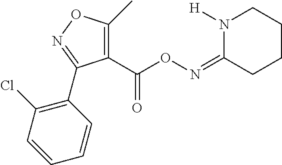 [SMILES: Cc1onc(-c2ccccc2Cl)c1C(=O)O/N=C1/CCCCN1] | D | Maybridge |

In vitro resistance studies performed with structure-related compounds further confirmed the antiviral activity of the lead series by selection of escape HCV cell colonies observed after 5 to 7 weeks of culture in the presence of compounds. HCV resistant variants were obtained that showed a shifted EC50>50 fold to HCV inhibitors, while maintaining sensitivity to previously reported NS3 protease and NS5A inhibitors. Complete HCV genome sequence obtained by deep sequencing with 500-7000 sequences reading at each nucleotide showed predominant mutations within HCV targets, which have never been reported for existing drug classes, validating a novel antiviral mechanism of action. Several mutations were also observed at lower frequency that map to other HCV proteins and are compatible with compensatory mechanism and associated high genetic barrier.

### REFERENCES

1. Raney, K. D., et al., Hepatitis C virus non-structural protein 3 (HCV NS3): a multifunctional antiviral target. J Biol Chem, 2010. 285(30): p. 22725-31.
2. Belon, C. A. and D. N. Frick, Helicase inhibitors as specifically targeted antiviral therapy for hepatitis C. Future Virol, 2009. 4(3): p. 277-293.
3. Lam, A. M. and D. N. Frick, Hepatitis C virus subgenomic replicon requires an active NS3 RNA helicase. J Virol, 2006. 80(1): p. 404-11.
4. Serebrov, V. and A. M. Pyle, Periodic cycles of RNA unwinding and pausing by hepatitis C virus NS3 helicase. Nature, 2004. 430(6998): p. 476-80.
5. Dumont, S., et al., RNA translocation and unwinding mechanism of HCV NS3 helicase and its coordination by ATP. Nature, 2006. 439(7072): p. 105-8.
6. Jennings, T. A., et al., NS3 helicase from the hepatitis C virus can function as a monomer or oligomer depending on enzyme and substrate concentrations. J Biol Chem, 2009. 284(8): p. 4806-14.
7. Sikora, B., et al., Hepatitis C virus NS3 helicase forms oligomeric structures that exhibit optimal DNA unwinding activity in vitro. J Biol Chem, 2008. 283(17): p. 11516-25.
8. Levin, M. K., Y. H. Wang, and S. S. Patel, The functional interaction of the hepatitis C virus helicase molecules is responsible for unwinding processivity. J Biol Chem, 2004. 279(25): p. 26005-12.
9. Liu, W. J., et al., Complementation analysis of the flavivirus Kunjin NS3 and NS5 proteins defines the minimal regions essential for formation of a replication complex and shows a requirement of NS3 in cis for virus assembly. J Virol, 2002. 76(21): p. 10766-75.
10. Ma, Y., et al., NS3 helicase domains involved in infectious intracellular hepatitis C virus particle assembly. J Virol, 2008. 82(15): p. 7624-39.
11. Beran, R. K. and A. M. Pyle, Hepatitis C viral NS3-4A protease activity is enhanced by the NS3 helicase. J Biol Chem, 2008. 283(44): p. 29929-37.
12. Beran, R. K., V. Serebrov, and A. M. Pyle, The serine protease domain of hepatitis C viral NS3 activates RNA helicase activity by promoting the binding of RNA substrate. J Biol Chem, 2007. 282(48): p. 34913-20.
13. Rajagopal, V., et al., The protease domain increases the translocation stepping efficiency of the hepatitis C virus NS3-4A helicase. J Biol Chem, 2010. 285(23): p. 17821-32.
14. Zhang, C., et al., Stimulation of hepatitis C virus (HCV) nonstructural protein 3 (NS3) helicase activity by the NS3 protease domain and by HCV RNA-dependent RNA polymerase. J Virol, 2005. 79(14): p. 8687-97.
15. Jennings, T. A., et al., RNA unwinding activity of the hepatitis C virus NS3 helicase is modulated by the NS5B polymerase. Biochemistry, 2008. 47(4): p. 1126-35.
16. Cheng, G., J. Zhong, and F. V. Chisari, Inhibition of dsRNA-induced signaling in hepatitis C virus-infected cells by NS3 protease-dependent and -independent mechanisms. Proc Natl Acad Sci USA, 2006. 103(22): p. 8499-504.
17. Ferreon, J. C., et al., Molecular determinants of TRIF proteolysis mediated by the hepatitis C virus NS3/4A protease. J Biol Chem, 2005. 280(21): p. 20483-92.
18. Li, K., et al., Immune evasion by hepatitis C virus NS3/4A protease-mediated cleavage of the Toll-like receptor 3 adaptor protein TRIF. Proc Natl Acad Sci USA, 2005. 102: p. 2992-7.
19. Li, X. D., et al., Hepatitis C virus protease NS3/4A cleaves mitochondrial antiviral signaling protein off the mitochondria to evade innate immunity. Proc Natl Acad Sci USA, 2005. 102(49): p. 17717-22.
20. Loo, Y. M., et al., Viral and therapeutic control of IFN-beta promoter stimulator 1 during hepatitis C virus infection. Proc Natl Acad Sci USA, 2006. 103(15): p. 6001-6.
21. Llinas-Brunet, M., et al., Peptide-based inhibitors of the hepatitis C virus serine protease. Bioorg Med Chem Lett, 1998. 8(13): p. 1713-8.
22. Steinkuhler, C., et al., Product inhibition of the hepatitis C virus NS3 protease. Biochemistry, 1998. 37(25): p. 8899-905.
23. Lamarre, D., et al., An NS3 protease inhibitor with antiviral effects in humans infected with hepatitis C virus. Nature, 2003. 426(6963): p. 186-9.
24. Chatel-Chaix, L., Germain, M A., Gotte, M. and Lamarre, D. Direct-acting and host-targeting HCV inhibitors: current and future directions. Curr Opin Virol. 2012 October; 2: p. 588-98.
25. Raney K D, Sharma S D, Moustafa I M, Cameron C E: Hepatitis C virus non-structural protein 3 (HCV NS3): a multifunctional antiviral target. J Biol Chem 2010, 285: p. 22725-22731.
26. Hanson A M, Hernandez J J, Shadrick W R, Frick D N: Identification and analysis of inhibitors targeting the hepatitis C virus NS3 helicase. Methods Enzymol 2012, 511:463-483
27. Frick D N: HCV Helicase: Structure, Function, and Inhibition. 2006.
28. Saalau-Bethell S M, et al., Discovery of an allosteric mechanism for the regulation of HCV NS3 protein function. Nat Chem Biol. 2012 November; 8(11): p. 920-5.

We claim:

1. A compound of Formula I 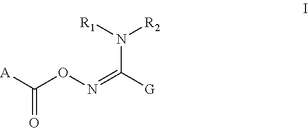
or a pharmaceutically acceptable salt thereof,
wherein
n is an integer of 1 or 2;
A is
1) C₁-C₇ alkyl,
2) C₃-C₇ alkyl,
3) C₁-C₇ alkyl-(aryl)ₙ,
4) (R³)(R⁴)C,
5) (R⁵)(R⁶)(R⁷)C,
6) aryl,
7) heteroaryl,
8) heterocyclyl, or
9) biphenyl,
wherein the aryl is substituted with one or more R¹⁰ substituents and the biphenyl is optionally substituted with one or more R¹⁰ substituents;
wherein the heteroaryl is substituted with one or more R²⁰ substituents; and
wherein the heterocyclyl is optionally substituted with C₁-C₇ alkyl, aryl-C₁-C₇ alkyl, the aryl moiety being optionally substituted with one or more R¹⁰ substituents;
G is
1) aryl,
2) C₁-C₇ alkyl-aryl,
3) heteroaryl, or
4) C₁-C₇ alkyl-heteroaryl,
wherein the aryl and the heteroaryl are optionally substituted with one or more R⁴⁰ substituents;
R¹ and R² are each independently
1) H,
2) C₁-C₇ alkyl,
3) C₃-C₇ cycloalkyl,
4) aryl,
5) heteroaryl, or
6) CH₂-heteroaryl,
wherein the aryl is optionally substituted with one or more R¹¹ substituents, and wherein the heteroaryl is optionally substituted with one or more R²⁰ substituents;
or
when R¹ is H, R² is covalently bonded to G to form a 3 to 7-membered heterocycle containing one or more heteroatoms selected from N, S or O;
R³, R⁴ when covalently bonded together form C₃-C₇ cycloalkyl or a heterocycle containing N or O heteroatoms optionally substituted with one or more R^{Y} substituents;
R⁵R⁶ and R⁷ are each independently
1) C₁-C₇ alkyl,
2) C₃-C₇ cycloalkyl, or
3) aryl optionally substituted with one or more R^{X} substituents;
R⁸ and R⁹ are both independently
1) H,
2) C₁-C₇ alkyl,
3) C₃-C₇ cycloalkyl,
4) C₁-C₇ alkyl C₃-C₇ cycloalkyl,
5) C(O)C₁-C₇alkyl,
6) C(O)C₃-C₇ cycloalkyl,
7) C(O)aryl,
8) C(O)heteroaryl
9) NHC(O)C₁-C₇ alkyl,
10) NHC(O)C₃-C₇ cycloalkyl,
11) NHC(O)aryl.
12) NHC(O)heteroaryl,
13) C₁-C₇ alkyl aryl, or
14) C₁-C₇ alkyl heteroaryl;
R¹⁰ is
1) halo,
2) CN,
3) OH,
4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹,
11) NHC(O),
12) C(O)OC₁-C₇ alkyl,
13) C(O)OH, or
14) aryl optionally substituted with one or more with R¹¹ substituents;
R¹¹ is
1) halo,
2) CN,
3) OH,
4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹;
11) NHC(O),
12) C(O)OC₁-C₇ alkyl, or
13) C(O)OH;
R²⁰ is
1) C₁-C₇ alkyl,
2) C₃-C₇ cycloalkyl,
3) aryl optionally substituted with one or more R¹⁰ substituents,
4) C₁-C₇ alkyl-aryl,
5) CH₂OCH₃, or
6) heteroaryl optionally substituted with one or more R¹⁰ substituents,
7) aryl-C₁-C₇ alkyl substituted with one or more R¹⁰ substituents,
8) heteroaryl-C₁-C₇ alkyl substituted with one or more R¹⁰ substituents;
wherein the aryl is optionally substituted with a halo substituent;
R⁴⁰ is
1) OR^{A},
2) halogen,
3) C₁-C₇ alkyl,
4) heteroaryl, or
5) haloalkane;
R^{A} is
1) C₁-C₇ alkane,
2) C₁-C₆ alkyl-aryl,
3) aryl, or
4) C₁-C₇ alkyl-heteroaryl,
wherein the aryl and the heteroaryl are optionally substituted with a halo;
R^{X} is
1) halo,
2) hydroxyl,
3) C₁-C₇ alkyl,
4) OC₁-C₇ alkyl; or
5) aryl optionally substituted with one or more R^{Z} substituents;
R^{Y} is
1) C₁-C₇ alkyl, or
2) aryl-C₁-C₇ alkyl optionally substituted with one or more R¹⁰ substituents; and
R^{Z} is
1) C₁-C₇ alkyl,
2) halo,
3) OH,
4) OC₁-C₇ alkyl, or
5) NR⁸R⁹;
and with the proviso that the following compounds are excluded:

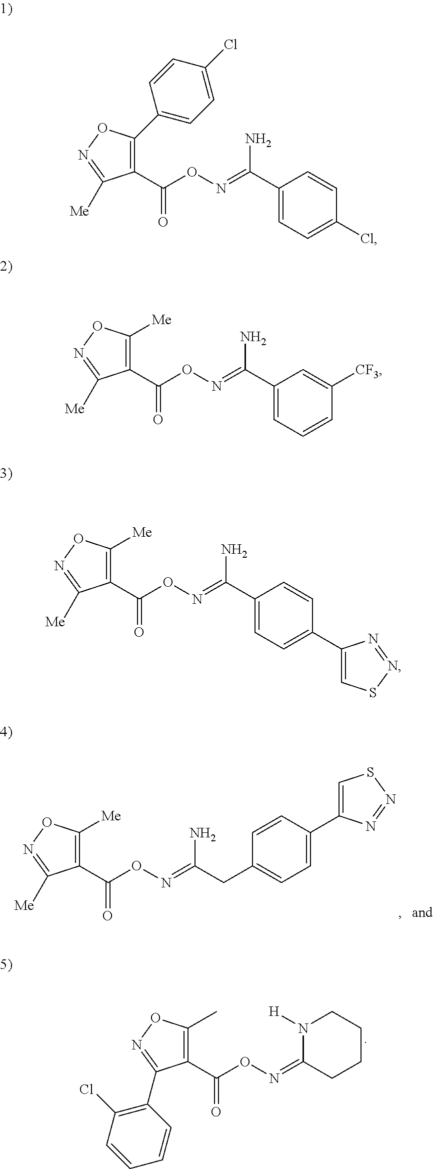 [SMILES: Cc1noc(-c2ccc(Cl)cc2)c1C(=O)O/N=C(\N)c1ccc(Cl)cc1 ; Cc1noc(C)c1C(=O)O/N=C(\N)c1cccc(C(F)(F)F)c1 ; Cc1noc(C)c1C(=O)O/N=C(\N)c1ccc(-c2csnn2)cc1 ; Cc1noc(C)c1C(=O)O/N=C(\N)Cc1ccc(-c2csnn2)cc1 ; Cc1onc(-c2ccccc2Cl)c1C(=O)O/N=C1/CCCCN1]

2. The compound, according to claim 1, in which includes a Core, the core being selected from one of the following Formulae 1A through 1H: 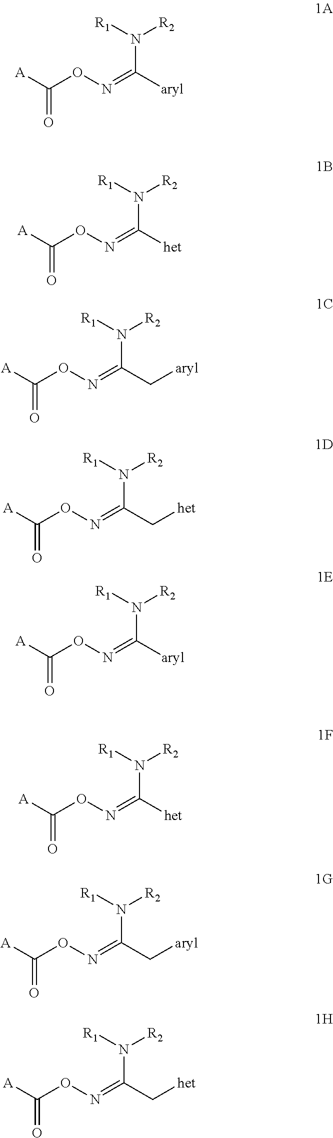
wherein the R¹, R², A, aryl and het are as defined in claim 1.

3. The compound, according to claim 1, in which A is
1) C₁-C₇ alkyl,
2) C₃-C₇ alkyl,
3) C₁-C₇ alkyl-(aryl)ₙ,
4) (R³)(R⁴)C,
5) (R⁵)(R⁶)(R⁷)C,
6) aryl,
7) heteroaryl,
8) heterocyclyl, or
9) biphenyl,
wherein the aryl is substituted with one or more R¹⁰ substituents and the biphenyl is optionally substituted with one or more R¹⁰ substituents;
wherein the heteroaryl is substituted with one or more R²⁰ substituents; and
wherein the heterocyclyl is optionally substituted with C₁-C₇ alkyl, aryl C₁-C₇ alkyl, the aryl moiety being optionally substituted with one or more R¹⁰ substituents.

4. The compound, according to claim 1, in which G is
1) aryl,
2) C₁-C₇ alkyl-aryl,
3) heteroaryl, or
4) C₁-C₇ alkyl-heteroaryl,
wherein the aryl and the heteroaryl are substituted with one or more R⁴⁰ substituents.

5. The compound, according to claim 1, in which R¹ and R² are each independently
1) H,
2) C₁-C₇ alkyl,
3) C₃-C₇ cycloalkyl,
4) aryl,
5) heteroaryl, or
6) CH₂-heteroaryl,
wherein the aryl is optionally substituted with one or more R¹¹ substituents, and wherein the heteroaryl is optionally substituted with one or more R²⁰ substituents; or
when R¹ is H, R² is covalently bonded to G to form a 3 to 7-membered heterocycle containing one or more heteroatoms selected from N, S or O.

6. The compound, according to claim 1, in which, R³, R⁴ when covalently bonded together form C₃-C₇ cycloalkyl or a heterocycle containing N or O heteroatoms optionally substituted with one or more R^{Y} substituents.

7. The compound, according to claim 1, in which R⁵R⁶ and R⁷ are each independently
1) C₁-C₇ alkyl,
2) C₃-C₇ cycloalkyl, or
3) aryl optionally substituted with one or more R^{X} substituents.

8. The compound, according to claim 1, in which R⁸ and R⁹ are both independently
1) H,
2) C₁-C₇ alkyl,
3) C₃-C₇ cycloalkyl,
4) C₁-C₇ alkyl-C₃-C₇ cycloalkyl,
5) C(O)C₁-C₇alkyl,
6) C(O)C₃-C₇ cycloalkyl,
7) C(O)aryl,
8) C(O)heteroaryl
9) NHC(O)C₁-C₇ alkyl,
10) NHC(O)C₃-C₇ cycloalkyl,
11) NHC(O)aryl.
12) NHC(O)heteroaryl,
13) C₁-C₇ alkyl aryl, or
14) C₁-C₇ alkyl heteroaryl.

9. The compound, according to claim 1, in which R¹⁰ is
1) halo,
2) CN,
3) OH,
4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹ mono or bis lower alkyl amino;
11) NHC(O),
12) C(O)OC₁-C₇ alkyl, or
13) C(O)OH, or
13) aryl optionally substituted with one or more with R¹¹ substituents.

10. The compound, according to claim 1, in which R¹¹ is
1) halo,
2) CN,
3) OH,
4) C₁-C₇ alkyl
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹;
11) NHC(O),
12) C(O)OC₁-C₇ alkyl, or
13) C(O)OH.

11. The compound, according to claim 1, in which R²⁰ is
1) C₁-C₇ alkyl,
2) C₃-C₇ cycloalkyl,
3) aryl optionally substituted with one or more R¹⁰ substituents,
4) C₁-C₇ alkyl-aryl,
5) CH₂OCH₃,
6) heteroaryl optionally substituted with one or more R¹⁰ substituents,
7) aryl C₁-C₇ alkyl substituted with one or more R¹⁰ substituents,
8) heteroaryl C₁-C₇ alkyl substituted with one or more R¹⁰ substituents;
wherein the aryl is optionally substituted with a halo substituent;

12. The compound, according to claim 1, in which R⁴⁰ is
1) OR^{A},
2) halogen,
3) C₁-C₇ alkyl,
4) heteroaryl, or
5) haloalkane.

13. The compound, according to claim 1, in which R^{A} is
1) C₁-C₇ alkyl,
2) C₁-C₆ alkyl-aryl,
3) aryl, or
4) C₁-C₇ alkyl-heteroaryl,
wherein the aryl and the heteroaryl are optionally substituted with a halo.

14. The compound, according to claim 1, in which R^{X} is
1) halo,
2) hydroxyl,
3) C₁-C₇ alkyl,
4) OC₁-C₇ alkyl; or
5) aryl optionally substituted with one or more R^{Z} substituents.

15. The compound, according to claim 1, in which R^{Y} is
1) C₁-C₇ alkyl, or
2) aryl C₁-C₇ alkyl optionally substituted with one or more R¹⁰ substituents.

16. The compound, according to claim 1, in which R^{Z} is
1) C₁-C₇ alkyl,
2) halo,
3) OH,
4) OC₁-C₇ alkyl, or
5) NR⁸R⁹.

17. A compound, according to claim 1, is selected from the group consisting of:

| Example # | Structure | |
|---|---|---|
| 1 | 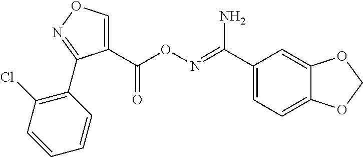 [SMILES: N/C(=N\OC(=O)c1conc1-c1ccccc1Cl)c1ccc2c(c1)OCO2] | ; |
| 2 | 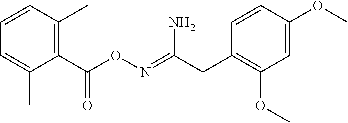 [SMILES: COc1ccc(C/C(N)=N/OC(=O)c2c(C)cccc2C)c(OC)c1] | ; |
| 3 | 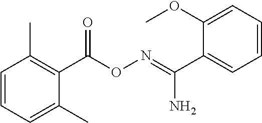 [SMILES: COc1ccccc1/C(N)=N/OC(=O)c1c(C)cccc1C] | -; |
| 4 | 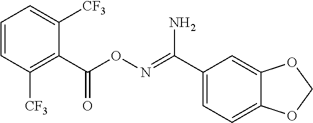 [SMILES: N/C(=N\OC(=O)c1c(C(F)(F)F)cccc1C(F)(F)F)c1ccc2c(c1)OCO2] | ; |
| 5 | 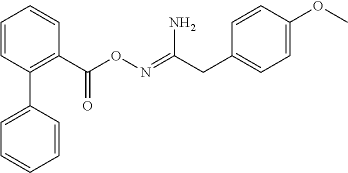 [SMILES: COc1ccc(C/C(N)=N/OC(=O)c2ccccc2-c2ccccc2)cc1] | ; |
| 6 | 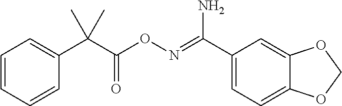 [SMILES: CC(C)(C(=O)O/N=C(\N)c1ccc2c(c1)OCO2)c1ccccc1] | ; |
| 7 | 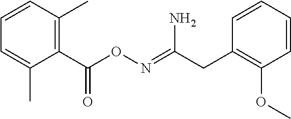 [SMILES: COc1ccccc1C/C(N)=N/OC(=O)c1c(C)cccc1C] | ; |
| 8 | 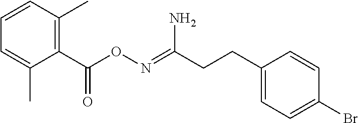 [SMILES: Cc1cccc(C)c1C(=O)O/N=C(\N)CCc1ccc(Br)cc1] | ; |
| 9 | 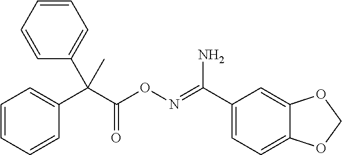 [SMILES: CC(C(=O)O/N=C(\N)c1ccc2c(c1)OCO2)(c1ccccc1)c1ccccc1] | ; |
| 10 | 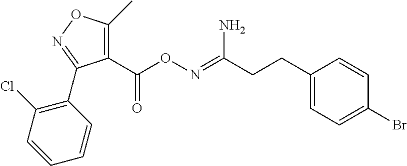 [SMILES: Cc1onc(-c2ccccc2Cl)c1C(=O)O/N=C(\N)CCc1ccc(Br)cc1] | ; |
| 11 | 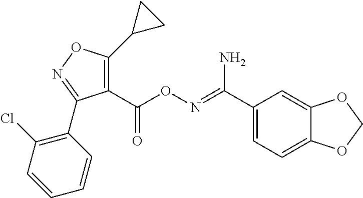 [SMILES: N/C(=N\OC(=O)c1c(-c2ccccc2Cl)noc1C1CC1)c1ccc2c(c1)OCO2] | ; |
| 12 | 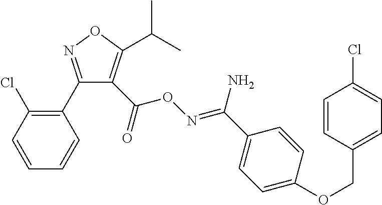 [SMILES: CC(C)c1onc(-c2ccccc2Cl)c1C(=O)O/N=C(\N)c1ccc(OCc2ccc(Cl)cc2)cc1] | ; |
| 13 | 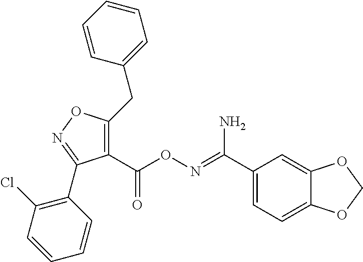 [SMILES: N/C(=N\OC(=O)c1c(-c2ccccc2Cl)noc1Cc1ccccc1)c1ccc2c(c1)OCO2] | ; |
| 14 | 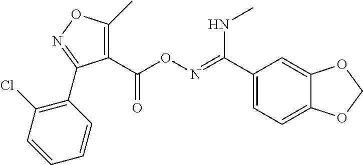 [SMILES: CN/C(=N\OC(=O)c1c(-c2ccccc2Cl)noc1C)c1ccc2c(c1)OCO2] | ; |
| 15 | 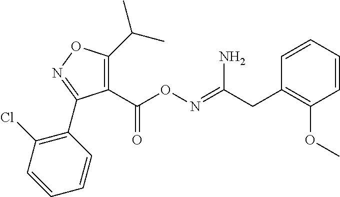 [SMILES: COc1ccccc1C/C(N)=N/OC(=O)c1c(-c2ccccc2Cl)noc1C(C)C] | ; |
| 16 | 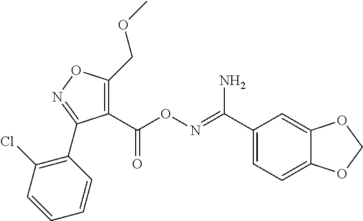 [SMILES: COCc1onc(-c2ccccc2Cl)c1C(=O)O/N=C(\N)c1ccc2c(c1)OCO2] | ; |
| 17 | 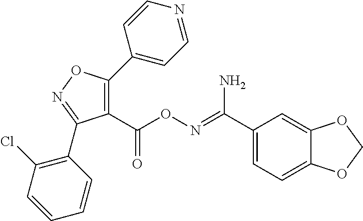 [SMILES: N/C(=N\OC(=O)c1c(-c2ccccc2Cl)noc1-c1ccncc1)c1ccc2c(c1)OCO2] | ; |
| 18 | 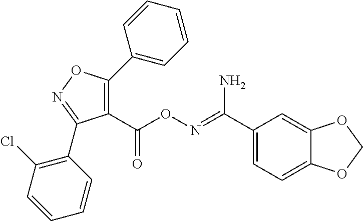 [SMILES: N/C(=N\OC(=O)c1c(-c2ccccc2Cl)noc1-c1ccccc1)c1ccc2c(c1)OCO2] | ; |
| 19 | 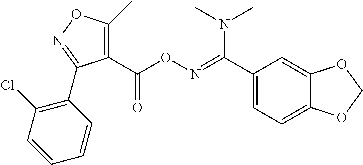 [SMILES: Cc1onc(-c2ccccc2Cl)c1C(=O)O/N=C(/c1ccc2c(c1)OCO2)N(C)C] | ; |
| 20 | 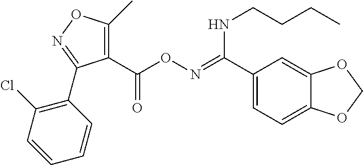 [SMILES: CCCCN/C(=N\OC(=O)c1c(-c2ccccc2Cl)noc1C)c1ccc2c(c1)OCO2] | ; |
| 21 | 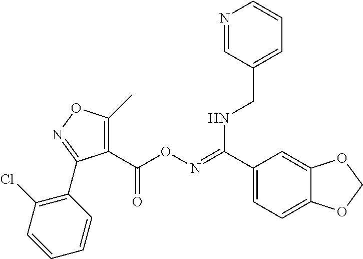 [SMILES: Cc1onc(-c2ccccc2Cl)c1C(=O)O/N=C(\NCc1cccnc1)c1ccc2c(c1)OCO2] | ; |
| 22 | 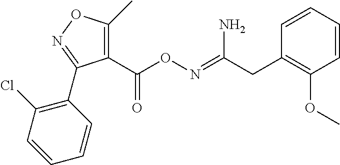 [SMILES: COc1ccccc1C/C(N)=N/OC(=O)c1c(-c2ccccc2Cl)noc1C] | ; |
| 23 | 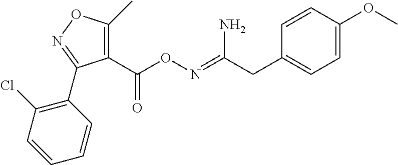 [SMILES: COc1ccc(C/C(N)=N/OC(=O)c2c(-c3ccccc3Cl)noc2C)cc1] | , |
| 24 | 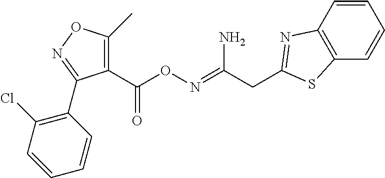 [SMILES: Cc1onc(-c2ccccc2Cl)c1C(=O)O/N=C(\N)Cc1nc2ccccc2s1] | ; |
| 25 | 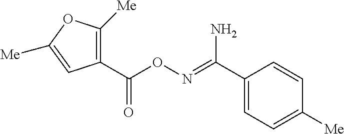 [SMILES: Cc1ccc(/C(N)=N/OC(=O)c2cc(C)oc2C)cc1] | ; |
| 26 | 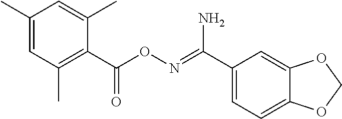 [SMILES: Cc1cc(C)c(C(=O)O/N=C(\N)c2ccc3c(c2)OCO3)c(C)c1] | ; |
| 27 | 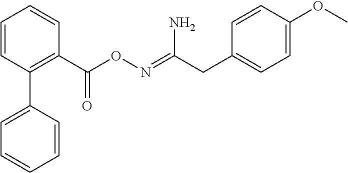 [SMILES: COc1ccc(C/C(N)=N/OC(=O)c2ccccc2-c2ccccc2)cc1] | ; |
| 28 | 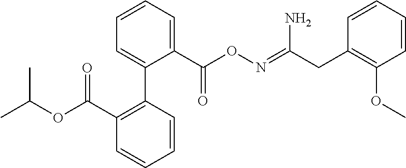 [SMILES: COc1ccccc1C/C(N)=N/OC(=O)c1ccccc1-c1ccccc1C(=O)OC(C)C] | ; |
| 29 | 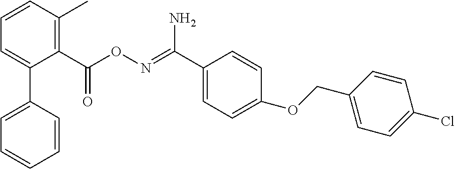 [SMILES: Cc1cccc(-c2ccccc2)c1C(=O)O/N=C(\N)c1ccc(OCc2ccc(Cl)cc2)cc1] | ; |
| 30 | 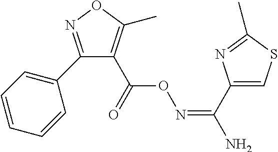 [SMILES: Cc1nc(/C(N)=N\OC(=O)c2c(-c3ccccc3)noc2C)cs1] | ; |
| 31 | 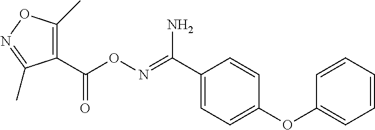 [SMILES: Cc1noc(C)c1C(=O)O/N=C(\N)c1ccc(Oc2ccccc2)cc1] | ; |
| 32 | 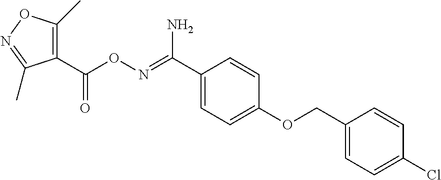 [SMILES: Cc1noc(C)c1C(=O)O/N=C(\N)c1ccc(OCc2ccc(Cl)cc2)cc1] | ; |
| 33 | 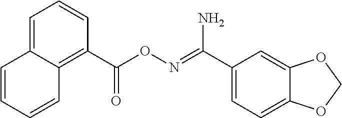 [SMILES: N/C(=N\OC(=O)c1cccc2ccccc12)c1ccc2c(c1)OCO2] | ; |
| 34 | 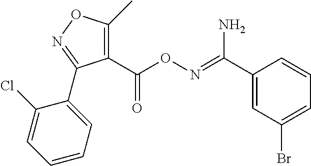 [SMILES: Cc1onc(-c2ccccc2Cl)c1C(=O)O/N=C(\N)c1cccc(Br)c1] | ; |
| 35 | 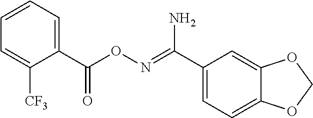 [SMILES: N/C(=N\OC(=O)c1ccccc1C(F)(F)F)c1ccc2c(c1)OCO2] | ; |
| 36 | 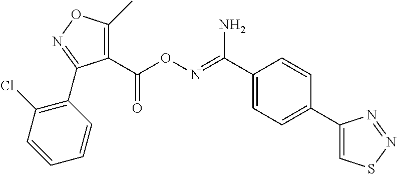 [SMILES: Cc1onc(-c2ccccc2Cl)c1C(=O)O/N=C(\N)c1ccc(-c2csnn2)cc1] | ; |
| 37 | 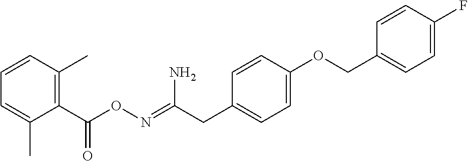 [SMILES: Cc1cccc(C)c1C(=O)O/N=C(\N)Cc1ccc(OCc2ccc(F)cc2)cc1] | ; |
| 38 | 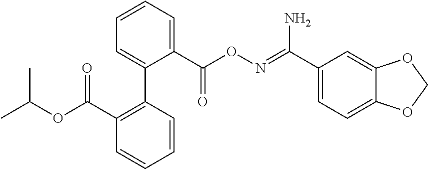 [SMILES: CC(C)OC(=O)c1ccccc1-c1ccccc1C(=O)O/N=C(\N)c1ccc2c(c1)OCO2] | ; |
| 39 | 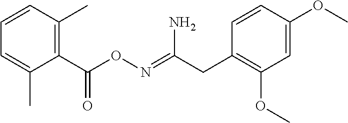 [SMILES: COc1ccc(C/C(N)=N/OC(=O)c2c(C)cccc2C)c(OC)c1] | ; |
| 40 | 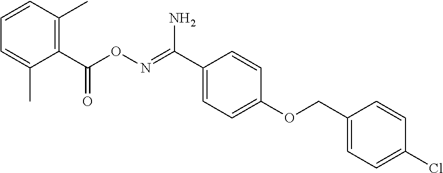 [SMILES: Cc1cccc(C)c1C(=O)O/N=C(\N)c1ccc(OCc2ccc(Cl)cc2)cc1] | ; |
| 41 | 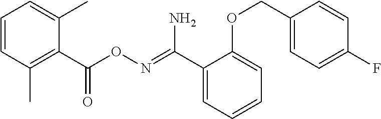 [SMILES: Cc1cccc(C)c1C(=O)O/N=C(\N)c1ccccc1OCc1ccc(F)cc1] | ; |
| 42 | 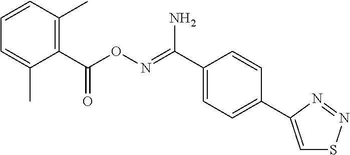 [SMILES: Cc1cccc(C)c1C(=O)O/N=C(\N)c1ccc(-c2csnn2)cc1] | ; |
| 43 | 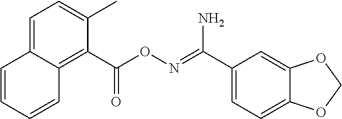 [SMILES: Cc1ccc2ccccc2c1C(=O)O/N=C(\N)c1ccc2c(c1)OCO2] | ; |
| 44 | 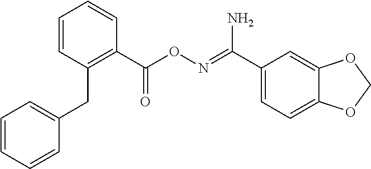 [SMILES: N/C(=N\OC(=O)c1ccccc1Cc1ccccc1)c1ccc2c(c1)OCO2] | ; |
| 45 | 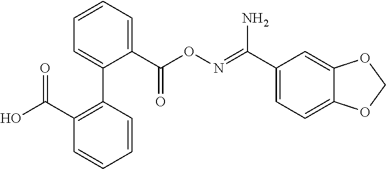 [SMILES: N/C(=N\OC(=O)c1ccccc1-c1ccccc1C(=O)O)c1ccc2c(c1)OCO2] | ; |
| 46 | 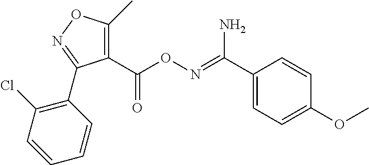 [SMILES: COc1ccc(/C(N)=N/OC(=O)c2c(-c3ccccc3Cl)noc2C)cc1] | ; |
| 47 | 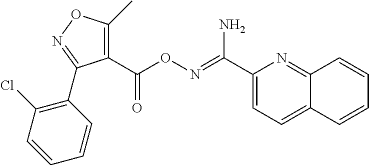 [SMILES: Cc1onc(-c2ccccc2Cl)c1C(=O)O/N=C(\N)c1ccc2ccccc2n1] | ; |
| 48 | 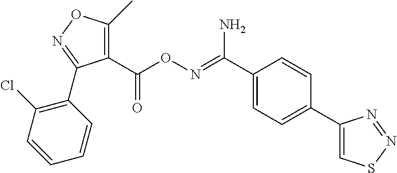 [SMILES: Cc1onc(-c2ccccc2Cl)c1C(=O)O/N=C(\N)c1ccc(-c2csnn2)cc1] | ; |
| 49 | 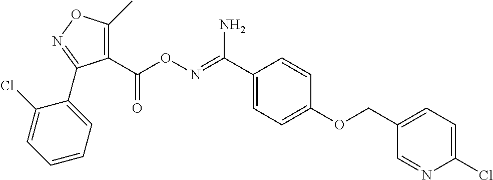 [SMILES: Cc1onc(-c2ccccc2Cl)c1C(=O)O/N=C(\N)c1ccc(OCc2ccc(Cl)nc2)cc1] | ; and |
| 50 | 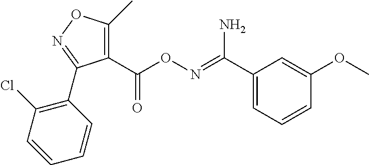 [SMILES: COc1cccc(/C(N)=N/OC(=O)c2c(-c3ccccc3Cl)noc2C)c1] | . |

18. A pharmaceutical composition comprising a compound of Formula 1, according to claim 1, and a pharmaceutically acceptable carrier.

19. A method of modulating or inhibiting dimerization of NS3/4A comprising:
containing a cell infected by HCV with a compound, according to Formula I: 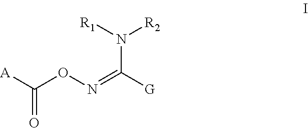
or a pharmaceutically acceptable salt thereof,
wherein
n is an integer of 1 or 2;
A is
1) C₁-C₇ alkyl,
2) C₃-C₇ alkyl,
3) C₁-C₇ alkyl-(aryl)ₙ,
4) (R³)(R⁴)C,
5) (R⁵)(R⁶)(R⁷)C,
6) aryl,
7) heteroaryl,
8) heterocyclyl, or
9) biphenyl,
wherein the aryl is substituted with one or more R¹⁰ substituents and the biphenyl is optionally substituted with one or more R¹⁰ substituents;
wherein the heteroaryl is substituted with one or more R²⁰ substituents; and
wherein the heterocyclyl is optionally substituted with C₁-C₇ alkyl, aryl-C₁-C₇ alkyl, the aryl moiety being optionally substituted with one or more R¹⁰ substituents;
G is
1) aryl,
2) C₁-C₇ alkyl-aryl,
3) heteroaryl, or
4) C₁-C₇ alkyl-heteroaryl,
wherein the aryl and the heteroaryl are optionally substituted with one or more R⁴⁰ substituents;
R¹ and R² are each independently
1) H,
2) C₁-C₇ alkyl,
3) C₃-C₇ cycloalkyl,
4) aryl,
5) heteroaryl, or
6) CH₂-heteroaryl,
wherein the aryl is optionally substituted with one or more R¹¹ substituents, and wherein the heteroaryl is optionally substituted with one or more R²⁰ substituents;
or
when R¹ is H, R² is covalently bonded to G to form a 3 to 7-membered heterocycle containing one or more heteroatoms selected from N, S or O;
R³, R⁴ when covalently bonded together form C₃-C₇ cycloalkyl or a heterocycle containing N or O heteroatoms optionally substituted with one or more R^{Y} substituents;
R⁵R⁶ and R⁷ are each independently
1) C₁-C₇ alkyl,
2) C₃-C₇ cycloalkyl, or
3) aryl optionally substituted with one or more R^{X} substituents;
R⁸ and R⁹ are both independently
1) H,
2) C₁-C₇ alkyl,
3) C₃-C₇ cycloalkyl,
4) C₁-C₇ alkyl C₃-C₇ cycloalkyl,
5) C(O)C₁-C₇alkyl,
6) C(O)C₃-C₇ cycloalkyl,
7) C(O)aryl,
8) C(O)heteroaryl
9) NHC(O)C₁-C₇ alkyl,
10) NHC(O)C₃-C₇ cycloalkyl,
11) NHC(O)aryl,
12) NHC(O)heteroaryl,
13) C₁-C₇ alkyl aryl, or
14) C₁-C₇ alkyl heteroaryl;
R¹⁰ is
1) halo,
2) CN,
3) OH,
4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹,
11) NHC(O),
12) C(O)OC₁-C₇ alkyl,
13) C(O)OH, or
14) aryl optionally substituted with one or more with R¹¹ substituents;
R¹¹ is
1) halo,
2) CN,
3) OH,
4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹;
11) NHC(O),
12) C(O)OC₁-C₇ alkyl, or
13) C(O)OH;
R²⁰ is
1) C₁-C₇ alkyl,
2) C₃-C₇ cycloalkyl,
3) aryl optionally substituted with one or more R¹⁰ substituents,
4) C₁-C₇ alkyl-aryl,
5) CH₂OCH₃,
6) heteroaryl optionally substituted with one or more R¹⁰ substituents,
7) aryl-C₁-C₇ alkyl substituted with one or more R¹⁰ substituents, or
8) heteroaryl-C₁-C₇ alkyl substituted with one or more R¹⁰ substituents;
wherein the aryl is optionally substituted with a halo substituent;
R⁴⁰ is
1) OR^{A},
2) halogen,
3) C₁-C₇ alkyl,
4) heteroaryl, or
5) haloalkane;
R^{A} is
1) C₁-C₇ alkane,
2) C₁-C₆ alkyl-aryl,
3) aryl, or
4) C₁-C₇ alkyl-heteroaryl,
wherein the aryl and the heteroaryl are optionally substituted with a halo;
R^{X} is
1) halo,
2) hydroxyl,
3) C₁-C₇ alkyl,
4) OC₁-C₇ alkyl; or
5) aryl optionally substituted with one or more R^{Z} substituents;
R^{Y} is
1) C₁-C₇ alkyl, or
2) aryl-C₁-C₇ alkyl optionally substituted with one or more R¹⁰ substituents; and
R^{Z} is
1) C₁-C₇ alkyl,
2) halo,
3) OH,
4) OC₁-C₇ alkyl, or
5) NR⁸R⁹, so as to modulate or inhibit dimerization of NS3/4A

20. A method of treating HCV infection in a subject, the method comprising:
administering to the subject in need thereof a therapeutically effective amount of a compound according to Formula I: 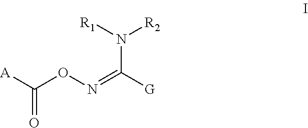
or a pharmaceutically acceptable salt thereof,
wherein
n is an integer of 1 or 2;
A is
1) C₁-C₇ alkyl,
2) C₃-C₇ alkyl,
3) C₁-C₇ alkyl-(aryl)ₙ,
4) (R³)(R⁴)C,
5) (R⁵)(R⁶)(R⁷)C,
6) aryl,
7) heteroaryl,
8) heterocyclyl, or
9) biphenyl,
wherein the aryl is substituted with one or more R¹⁰ substituents and the biphenyl is optionally substituted with one or more R¹⁰ substituents;
wherein the heteroaryl is substituted with one or more R²⁰ substituents; and
wherein the heterocyclyl is optionally substituted with C₁-C₇ alkyl, aryl-C₁-C₇ alkyl, the aryl moiety being optionally substituted with one or more R¹⁰ substituents;
G is
1) aryl,
2) C₁-C₇ alkyl-aryl,
3) heteroaryl, or
4) C₁-C₇ alkyl-heteroaryl,
wherein the aryl and the heteroaryl are optionally substituted with one or more R⁴⁰ substituents;
R¹ and R² are each independently
1) H,
2) C₁-C₇ alkyl,
3) C₃-C₇ cycloalkyl,
4) aryl,
5) heteroaryl, or
6) CH₂-heteroaryl,
wherein the aryl is optionally substituted with one or more R¹¹ substituents, and wherein the heteroaryl is optionally substituted with one or more R²⁰ substituents;
or
when R¹ is H, R² is covalently bonded to G to form a 3 to 7-membered heterocycle containing one or more heteroatoms selected from N, S or O;
R³, R⁴ when covalently bonded together form C₃-C₇ cycloalkyl or a heterocycle containing N or O heteroatoms optionally substituted with one or more R^{Y} substituents;
R⁵R⁶ and R⁷ are each independently
1) C₁-C₇ alkyl,
2) C₃-C₇ cycloalkyl, or
3) aryl optionally substituted with one or more R^{X} substituents;
R⁸ and R⁹ are both independently
1) H,
2) C₁-C₇ alkyl,
3) C₃-C₇ cycloalkyl,
4) C₁-C₇ alkyl C₃-C₇ cycloalkyl,
5) C(O)C₁-C₇alkyl,
6) C(O)C₃-C₇ cycloalkyl,
7) C(O)aryl,
8) C(O)heteroaryl
9) NHC(O)C₁-C₇ alkyl,
10) NHC(O)C₃-C₇ cycloalkyl,
11) NHC(O)aryl.
12) NHC(O)heteroaryl,
13) C₁-C₇ alkyl aryl, or
14) C₁-C₇ alkyl heteroaryl;
R¹⁰ is
1) halo,
2) CN,
3) OH,
4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹,
11) NHC(O),
12) C(O)OC₁-C₇ alkyl,
13) C(O)OH, or
14) aryl optionally substituted with one or more with R¹¹ substituents;
R¹¹ is
1) halo,
2) CN,
3) OH,
4) C₁-C₇ alkyl,
5) C₃-C₇ cycloalkyl,
6) OC₁-C₇ alkyl,
7) SC₁-C₇ alkyl,
8) haloalkyl,
9) C₁-C₇ alkyl-aryl,
10) NR⁸R⁹;
11) NHC(O),
12) C(O)OC₁-C₇ alkyl, or
13) C(O)OH;
R²⁰ is
1) C₁-C₇ alkyl,
2) C₃-C₇ cycloalkyl,
3) aryl optionally substituted with one or more R¹⁰ substituents,
4) C₁-C₇ alkyl-aryl,
5) CH₂OCH₃,
6) heteroaryl optionally substituted with one or more R¹⁰ substituents,
7) aryl-C₁-C₇ alkyl substituted with one or more R¹⁰ substituents, or
8) heteroaryl-C₁-C₇ alkyl substituted with one or more R¹⁰ substituents;
wherein the aryl is optionally substituted with a halo substituent;
R⁴⁰ is
1) OR^{A},
2) halogen,
3) C₁-C₇ alkyl,
4) heteroaryl, or
5) haloalkane;
R^{A} is
1) C₁-C₇ alkane,
2) C₁-C₆ alkyl-aryl,
3) aryl, or
4) C₁-C₇ alkyl-heteroaryl,
wherein the aryl and the heteroaryl are optionally substituted with a halo;
R^{X} is
1) halo,
2) hydroxyl,
3) C₁-C₇ alkyl,
4) OC₁-C₇ alkyl; or
5) aryl optionally substituted with one or more R^{Z} substituents;
R^{Y} is
1) C₁-C₇ alkyl, or
2) aryl-C₁-C₇ alkyl optionally substituted with one or more R¹⁰ substituents; and
R^{Z} is
1) C₁-C₇ alkyl,
2) halo,
3) OH,
4) OC₁-C₇ alkyl, or
5) NR⁸R⁹, so as to treat the HCV infection.
